(19) United States
(12) Patent Application Publication
(10) Pub. No.: US 2010/0285516 A1
(43) Pub. Date: Nov. 11, 2010

(54) CALCIUM FLUX AS A PHARMACOEFFICACY BIOMARKER FOR INHIBITORS OF HISTONE DEACETYLASE

(75) Inventors: Sriram Balasubramanian, San Carlos, CA (US); Joseph J. Buggy, Mountain View, CA (US); Jason Ramos, San Leandro, CA (US); Mint Sirisawad, Mountain View, CA (US)

Correspondence Address: WILSON, SONSINI, GOODRICH & ROSATI, 650 PAGE MILL ROAD, PALO ALTO, CA, 94304-1050, US

(73) Assignee: PHARMACYCLICS, INC., Sunnyvale, CA (US)

(21) Appl. No.: 12/595,802

(22) PCT Filed: Apr. 11, 2008
(86) PCT No.: PCT/US2008/060098

§ 371 Date: Jul. 9, 2010

Related U.S. Application Data
(63) Continuation-in-part of application No. 11/779,743, filed on Jul. 18, 2007.
(60) Provisional application No. 60/911,857, filed on Apr. 13, 2007.
(60) Provisional application No. 60/944,409, filed on Jun. 15, 2007.
(60) Provisional application No. 60/954,777, filed on Aug. 8, 2007.
(60) Provisional application No. 61/026,023, filed on Feb. 4, 2008.

(51) Int. Cl. C12Q 1/02

(52) U.S. Cl. 435/29

(57) ABSTRACT

Described herein are methods for using calcium flux as a biomarker to select and predict patients likely to respond to an apoptotic agent as therapy. Further described herein is a method of using calcium flux as a clinical biomarker to determine whether a tumor is sensitive to an HDAC inhibitor.

### RELATED APPLICATIONS

This application claims the benefit of U.S. Provisional Patent Application No. 60/911,857 filed Apr. 13, 2007, U.S. Provisional Patent Application No. 60/944,409 filed Jun. 15, 2007, U.S. Provisional Patent Application No. 60/954,777 filed Aug. 8, 2007, U.S. Provisional Patent Application No. 61/026,023 filed Feb. 4, 2008, and U.S. Nonprovisional patent application Ser. No. 11/779,743 filed Jul. 18, 2007; the disclosures of these references are herein incorporated in their entirety.

### FIELD OF INVENTION

Described herein are methods for using calcium flux as a biomarker to select and predict patients likely to respond to treatment with an inhibitor of histone deacetylase.

### BACKGROUND

Biomarkers are substances that can be used as indicators of a biologic state. For example, a biomarker can be a substance, the presence of which is indicative of the presence of a disease. They are characteristics that may be objectively measured and evaluated as an indicator of normal biological processes, pathogenic processes, or pharmacologic responses to a therapeutic intervention. In the clinical setting, biomarkers are observable features or detectable substances that correlate well with a disease state or therapeutic outcome and thus can be used to measure the progress of disease or the effects of treatment.

### SUMMARY OF THE INVENTION

Described herein is use of calcium flux as a biomarker. Also described herein are rapid methods for assessing the ability of an agent to cause apoptosis. Further described herein are methods for using calcium flux as a biomarker to select and predict patients likely to respond to an apoptotic agent as therapy.

Some embodiments comprise a method of selecting a patient with a condition which responds to treatment with an apoptotic agent comprising the steps of: contacting a biological sample from a subject with an apoptotic agent; measuring the level of calcium flux in the biological sample, and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the apoptotic agent is selected from the group consisting of: a pan-HDAC inhibitor and an HDAC8 inhibitor. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample is a blood sample. In some embodiments, the biological sample comprises at least about 100 tumor cells.

Some embodiments comprise a method of selecting a patient with a condition which responds to treatment with an apoptotic agent comprising the steps of: contacting a biological sample from a subject with a pan-HDAC inhibitor; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a method of selecting a patient with a condition which responds to treatment with an apoptotic agent comprising the steps of: contacting a biological sample from a subject with an HDAC8 inhibitor; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting ofL Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a method of selecting a patient for participation in a clinical trial to evaluate the efficacy of an apoptotic agent in treating a condition comprising the steps of: contacting a biological sample from a subject with the apoptotic agent; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the apoptotic agent is selected from the group consisting of: a pan-HDAC inhibitor and an HDAC8 inhibitor. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a system for selecting a patient with a condition which responds to treatment with an apoptotic agent comprising: (a) an apoptotic agent; (b) a means for measuring the level of calcium flux in the biological sample; wherein the biological sample, apoptotic agent, and means for measuring the level of calcium flux are all in fluidic communication. In some embodiments, the apoptotic agent is selected from the group consisting of: a pan-HDAC inhibitor and an HDAC8 inhibitor. In some embodiments, the biological sample comprises tumor cells. In some embodiments, the means for measuring the level of calcium flux comprises a calcium detection reagent.

Optionally, the biopsy is obtained from a tumor or blood taken from a patient. Tumor samples include all tumors, both solid and liquid. In some embodiments, the biopsy is obtained from circulating tumor cells (CTCs) isolated from blood of patients with solid tumors. In certain embodiments, the biopsy is performed with at least about 10² tumor cells.

Optionally, the calcium flux assay works with clinical samples obtained via fine needle aspiration (FNA), punch biopsy; a biopsy is obtained from tissue, cells, or fluids from a living body; or biopsies are performed by a biopsy needle or by open surgical incision. In some embodiments, the biopsy is treated ex vivo or in vitro with a sufficient concentration of an apoptotic agent.

### DEFINITIONS

Unless otherwise stated, the following terms used in the specification and claims are defined for the purposes of this application and have the following meanings:

As used herein, an “apoptotic agent” is any agent that directly or indirectly induces apoptosis in a cell. By way of nonlimiting example, the apoptotic agent may be an HDAC inhibitor, a cytotoxic agent, a kinase inhibitor, or an antibody. In some embodiments, an apoptotic agent is selected from a pan-HDAC inhibitor or an HDAC8 inhibitor.

As used herein, “calcium detection reagent” means any chemical or biological agent that may be used alone or in combination with one or more agents to indicate intracellular calcium levels. By way of nonlimiting example, the calcium detection reagent may be a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof.

As used herein, “calcium flux” means the movement of calcium ions across cellular membranes, between organelles, or through the cytoplasm.

As used herein, “derivative” means a compound that is produced from another compound or similar structure by the replacement or substitution of an atom, molecule, or group by another atom, molecule, or group. By way of nonlimiting example, if a pan-HDAC inhibitor or a HDAC8 inhibitor contains an oxidizable nitrogen atom, the nitrogen atom may be converted to an N-oxide by known methods to produce an N-oxide derivative. By way of further nonlimiting example, if a pan-HDAC inhibitor or HDAC8 inhibitor contains a hydroxy group, a carboxy group, a thiol group, or any group containing one or more nitrogen atoms these groups may be protected with suitable protecting groups to produce a protected derivative. A list of suitable protective groups is found in T. W. Greene, Protective Groups in Organic Synthesis, John Wiley & Sons, Inc. 1981, which is incorporated herein by reference.

As used herein, “effective amount,” or “therapeutically effective amount” means an amount of an agent which confers a pharmacological effect or therapeutic effect on a subject without undue adverse side effects. It is understood that the effective amount or the therapeutically effective amount will vary from subject to subject, based on the subject's age, weight, and general condition, the condition being treated, the severity of the condition being treated, and the judgment of the prescribing physician.

As used herein, “fluorophore” means a molecule which, when excited, emits photons. By way of nonlimiting example, the fluorophore may be Fura-2, Indo-1, Fluo-3, calcein, Rhod-2, Rhod-4, and derivatives thereof.

As used herein, “histone deacetylase” and “HDAC” refer to any one of a family of enzymes that remove acetyl groups from the ε-amino groups of lysine residues at the N-terminus of a histone. Unless otherwise indicated, the term “histone” means any histone protein, including H1, H2A, H2B, H3, H4, and H5, from any species. Human HDAC proteins or gene products, include, but are not limited to, HDAC-1, HDAC-2, HDAC-3, HDAC4, HDAC-5, HDAC-6, HDAC-7, HDAC-8, HDAC-9, HDAC-10, and HDAC-11. In some embodiments, the HDAC is also derived from a protozoal or fungal source.

As used herein, “histone deacetylase inhibitor,” “inhibitor of histone deacetylase,” “HDAC inhibitor,” and “inhibitor of HDAC” are used interchangeably to identify a compound, which is capable of interacting with a HDAC and inhibiting its activity, more particularly its enzymatic activity. Inhibiting HDAC enzymatic activity means reducing the ability of a HDAC to remove an acetyl group from a histone. In some embodiments, such inhibition is specific, i.e. the HDAC inhibitor reduces the ability of a HDAC to remove an acetyl group from a histone at a concentration that is lower than the concentration of the inhibitor that is required to produce some other, unrelated biological effect.

As used herein, “HDAC8 inhibitor,” and “inhibitor of HDAC8” are used interchangeably to identify a compound, which is capable of interacting with an HDAC8 enzyme and inhibiting its activity, more particularly its enzymatic activity. Inhibiting HDAC8 enzymatic activity means reducing the ability of a HDAC8 to remove an acetyl group from a protein or other macromolecule.

As used herein, “pan-HDAC” inhibitor is (a) a chemical or biological agent that inhibits all eleven HDAC isoforms of the class I and class II enzymes, or (b) a chemical or biological agent that significantly inhibits more that one isoform of class I or class II HDACs with a Ki of less than 1 μM.

As used herein, “prodrug” means a drug or compound in which the pharmacological action results from conversion by metabolic processes within the body. Prodrugs are generally drug precursors that, following administration to a subject and subsequent absorption, are converted to an active or more active species via some process, such as conversion by metabolic pathway. Some prodrugs have a chemical group present on the prodrug which renders it less active and/or confers solubility or some other property to the drug. Once the chemical group has been cleaved and/or modified from the prodrug, the active drug is generated.

As used herein, “treat,” “treating,” or “treatment” refers to, but is not limited to, inhibiting the progression of a disorder or disease, for example arresting the development of the disease or disorder. By way of nonlimiting example, treatment of cancer includes the induction of apoptosis in malignant cells, or any effect that results in the inhibition of the growth of the malignant cells and/or of the ability of the malignant cells to metastisize.

### DESCRIPTION OF DRAWINGS

FIG. 1 shows a schematic representation of apoptosis induction by an outside agent that would involve PLCγ1 and downstream calcium signaling

FIG. 2 shows rapid induction of calcium flux by different HDAC inhibitors in Jurkat cells: (A) Control; (B) 0.2 μM pan HDAC Inhibitor PCI-24781 and percentage of apoptosis as measured by Annexin-V after 2-day treatments with PCI-24781: 80%; (C) 10 μM MACS inhibitor PCI-34051 and percentage of apoptosis as measured by Annexin-V after 2-day treatments with PCI-34051: 60%; (D) 1 μM MS-275; (E) 1 mM NaButyrate; and (F) 2 μM SAHA. A calcium flux response correlates with a high percentage of apoptosis.

FIG. 3 shows no induction of calcium flux by either pan-HDAC inhibitor compound PCI-24781 and HDAC8 inhibitor compound PCI-34051 in HH cells (lacking active PLCγ enzyme). Also shown are the percentages of apoptosis as measured by Annexin-V after 2-day treatments with PCI-24781 and PCI-34051. A calcium flux response correlates with a high percentage of apoptosis. (A) Control; (B) 10 μM HDAC8 inhibitor PCI-34051; (C) 0.2 μM pan-HDAC inhibitor PCI-24781.

FIG. 4 is a bar graph showing the ability of an HDAC8 inhibitor compound (PCI-34051; 5 μM) to effect apoptosis in Jurkat cells that are wild-type (Jurkat); phospholipase C-γ1 deficient (J.gamma1); T-cell receptor-deficient (P116); or ZAP-70-deficient (JRT3-T.5). Jurkat cells are human T-cell leukemia established from the peripheral blood of a 14 year old boy with acute lymphoblastic leukemia.

FIG. 5 shows the effect of calcium mobilization when a Phospholipase C inhibitor is added to Jurkat cells with and without an HDAC inhibitor. (A) Control. (B) HDAC8 inhibitor PCI-34051. (C) Shows rapid induction of calcium mobilization in Jurkat cells by PCI-34051 is inhibited when a Phospholipase C inhibitor (U-73122: 1-[6-((17b-3-Methoxyestra-1,3,5(10)-trien-17-yl)amino)hexyl]-1H-pyrrole-2,5-dione) is added. (D) The inactive analog inhibitor of PLC (U-73343: 1-[6-((17b-3-Methoxyestra-1,3,5(10)-trien-17-yl)amino)hexyl]-2,5-pyrrolidinedione) had no effect on the calcium mobilization induced by PCI-34051. (E-F) Neither PLCγinhibitor (U-73343, U-73122) caused induction of calcium flux alone.

FIG. 6 is a bar graph showing a PLC inhibitor modulates PCI-34051-induced apoptosis in Jurkat cells. Jurkat and J.gamma1 cells treated with PLC inhibitor U-73122 and inactive analog U-73343 with or without PCI-34051 and Annexin-V measured after 2 days.

FIG. 7 is a bar graph that shows the effect of a calcium effector (thapsigargin; 0.2 μM) on the induction of apoptosis by PCI-34051 (10 μM) in wild-type (Jurkat) versus J.γ1 (J.gamma1) Jurkat cells.

FIG. 8(A) is a bar graph that shows the effect of a calcium chelator (BAPTA-AM; 0.5 μM) on the induction of apoptosis by PCI-34051 (10 μM) in wild-type (Jurkat) versus J.γ1 (J.gamma1) Jurkat cells. (B) is a line graph that shows PCI-34051-induced calcium flux in Jurkat cells is blocked by the cell permeable calcium chelator BAPTA-AM.

FIG. 9 shows a series of immunoblot images demonstrating the translocation of cytochrome C oxidase translocation from mitochondria to cytosol in wild-type (Jurkat) versus J.γ1 (J.gamma1) Jurkat cells at various time points following treatment with pro-apoptotic agents.

FIG. 10 is a line graph showing dose-dependent intracellular calcium responses induced by a HDAC8-selective inhibitor compound (PCI-34051) in Jurkat cells.

FIG. 11 is a line graph showing dose-dependent intracellular calcium responses induced by a pan-HDAC inhibitor compound (PCI-24781) in Jurkat cells.

FIG. 12(A) shows induction of calcium flux by PCI-24781, but not PCI-34051 in Ramos cells. Also shown are the percentages of apoptosis as measured by Annexin-V after 2-day treatments with PCI-24781 and PCI-34051; (B) shows no induction of calcium flux by either PCI-24781 or PCI-34051 in J.gamma1 cells. Also shown are the percentages of apoptosis as measured by Annexin-V after 2-day treatments with PCI-24781 and PCI-34051; (C) shows induction of calcium flux by PCI-24781 but not PCI-34051 in HCT-116 colon cells. Also shown are the percentages of apoptosis as measured by Annexin-V after 2-day treatments with PCI-24781 and PCI-34051; (D) shows no induction of calcium flux by either PCI-24781 or PCI-34051 in PC3 prostate cells. Also shown are the percentages of apoptosis as measured by Annexin-V staining after two-day treatments with PCI-24781 and PCI-34051 in those cell lines. In all figures a calcium flux response correlates with a high percentage of apoptosis.

FIG. 13 shows induction of calcium flux by PCI-24781, but not PCI-34051 in A549 cells. The lung tumor line A549 is representative of an epithelial solid tumor line that responds to the pan-HDAC inhibitor, PCI-24781, by induction of apoptosis which is predicted by calcium flux, while the T-cell specific HDAC8 selective inhibitor PCI-34051 neither induces apoptosis nor calcium flux in these cells. Also shown are the percentages of apoptosis as measured by Annexin-V after 2-day treatments with PCI-24781 and PCI-34051;

FIG. 14 shows induction of calcium flux by PCI-24781, but not PCI-34051 in THP-1 cells. THP-1 is a monocytic leukemia line in which the pan-HDAC inhibitor PCI-24781 can induce apoptosis and calcium flux, whereas the HDAAC8 selective inhibitor PCI-34051 does not. Also shown are the percentages of apoptosis as measured by Annexin-V after 2-day treatments with PCI-24781 and PCI-34051;

FIG. 15 shows rapid induction of calcium mobilization in Jurkat cells by PCI-24781. Two major metabolites of PCI-24781, the carboxylic acid metabolite PCI-27789 and the amide metabolite PCI-27787, have no histone deacetylase activity. PCI-27789 and PCI-27787 were not able to cause induction of calcium flux in Jurkat cells.

FIG. 16 shows induction of apoptosis by PCI-24781, while the inactive metabolites of PCI-24781, PCI-27789 and PCI-27787, have no apoptosis-inducing effect in Jurkat cells. Jurkat cells treated with PCI-24781, PCI-27789 or PCI-27787 (0.2 uM) and apoptosis measured by Annexin-V staining after 2 days.

FIG. 17 shows rapid induction of calcium flux by PCI-24781 and PCI-34051 in a Cutaneous T-cell Lymphoma patient biopsy (SF-03). (A) Control; (B) 0.2 μM PCI-24781; (C) 10 μM PCI-34051.

FIG. 18 shows no induction of calcium flux by PCI-24781 or PCI-34051 in a Cutaneous T-cell Lymphoma patient biopsy (SF-06). (A) Control; (B) 0.2 μM PCI-24781; (C) 10 μM PCI-34051.

### DETAILED DESCRIPTION OF THE INVENTION

Some embodiments comprise a method of selecting a patient with a condition which responds to treatment with an apoptotic agent comprising the steps of: contacting a biological sample from a subject with an apoptotic agent; measuring the level of calcium flux in the biological sample, and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the apoptotic agent is selected from the group consisting of: a pan-HDAC inhibitor and an HDAC8 inhibitor. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample is a blood sample. In some embodiments, the biological sample comprises at least about 100 tumor cells.

Some embodiments comprise a method of selecting a patient with a condition which responds to treatment with an apoptotic agent comprising the steps of: contacting a biological sample from a subject with a pan-HDAC inhibitor; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a method of selecting a patient with a condition which responds to treatment with an apoptotic agent comprising the steps of: contacting a biological sample from a subject with an HDAC8 inhibitor; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the level of calcium flux is measured with a calcium detection reagent. In some embodiments, the calcium detection reagent is a fluorophore. In some embodiments, the fluorophore is selected from the group consisting ofL Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a method of selecting a patient for participation in a clinical trial to evaluate the efficacy of an apoptotic agent in treating a condition comprising the steps of: contacting a biological sample from a subject with the apoptotic agent; measuring the level of calcium flux in the biological sample; and selecting the patient for treatment with the apoptotic agent if the level of calcium flux observed in the biological sample following contact with the apoptotic agent exceeds the level of calcium flux observed in a control, or the biological sample before contact with the apoptotic agent; or selecting an alternative treatment if the level of calcium flux observed in the biological sample following contact with the apoptotic agent does not exceed the level of calcium flux observed in a control or the biological sample before contact with the apoptotic agent. In some embodiments, the apoptotic agent is selected from the group consisting of: a pan-HDAC inhibitor and an HDAC8 inhibitor. In some embodiments, the level of calcium flux is measured with a calcium detection reagent. In some embodiments, the calcium detection reagent is a fluorophore. In some embodiments, the fluorophore is selected from the group consisting of: Fura-2, Indo-1, Fluo-3, calcein, Rhod-2, Rhod-4, and derivatives thereof. In some embodiments, the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas. In some embodiments, the biological sample comprises tumor cells. In some embodiments, the biological sample comprises circulating tumor cells obtained from a blood sample. In some embodiments, the biological sample comprises at least about 100 tumor cells.

Some embodiments comprise a system for selecting a patient with a condition which responds to treatment with an apoptotic agent comprising: (a) an apoptotic agent; (b) a means for measuring the level of calcium flux in the biological sample; wherein the biological sample, apoptotic agent, and means for measuring the level of calcium flux are all in fluidic communication. In some embodiments, the apoptotic agent is selected from the group consisting of: a pan-HDAC inhibitor and an HDAC8 inhibitor. In some embodiments, the biological sample comprises tumor cells. In some embodiments, the means for measuring the level of calcium flux comprises a calcium detection reagent.

The Phosphoinositide phospholipase C (PLC) family and Ca-mediated signaling

The phosphoinositide phospholipase C (PLC) family is a family of eukaryotic enzymes that participate in signal transduction. The PLC family consists of six sub-families comprising a total of 13 separate isoforms. PLCγ participates in T-cell responses to external stimuli (such as, growth factors, neurotransmitters) and internal stimuli (for example, PI3K).

Members of the phospholipase C (PLC) family have been shown to catalyze the hydrolysis of PIP₂, a phosphatidylinositol, generating two second messengers, inositol triphosphate (IP₃) and diacylglycerol (DAG). The hydrolysis of PIP₂ occurs in two sequential steps. The first reaction is a phosphotranseferase step that involves an intramolecular attack between the hydroxyl group in the 2′ position on the inositol ring and the phosphate group resulting in a cyclic IP₃ intermediate. At this point DAG is generated. However, in the second phosphodiesterase step, the cyclic intermediate is held within the active site long enough to be attacked by a molecule of water resulting in a final acyclic IP₃ product.

IP₃ and DAG modulate the activity of downstream proteins important for cellular signaling. IP₃ is soluble and diffuses through the cytoplasm and interacts with IP₃ receptors on the endoplasmic reticulum (ER), causing the release of calcium and raising the level of intracellular calcium (one source of calcium flux). DAG remains tethered to the inner leaflet of the plasma membrane, due to its hydrophobic character, where it recruits protein kinase C (PKC). PKC becomes activated in when bound to calcium ions. The activation of PKC results in a multitude of cellular responses through stimulation of calcium sensitive proteins.

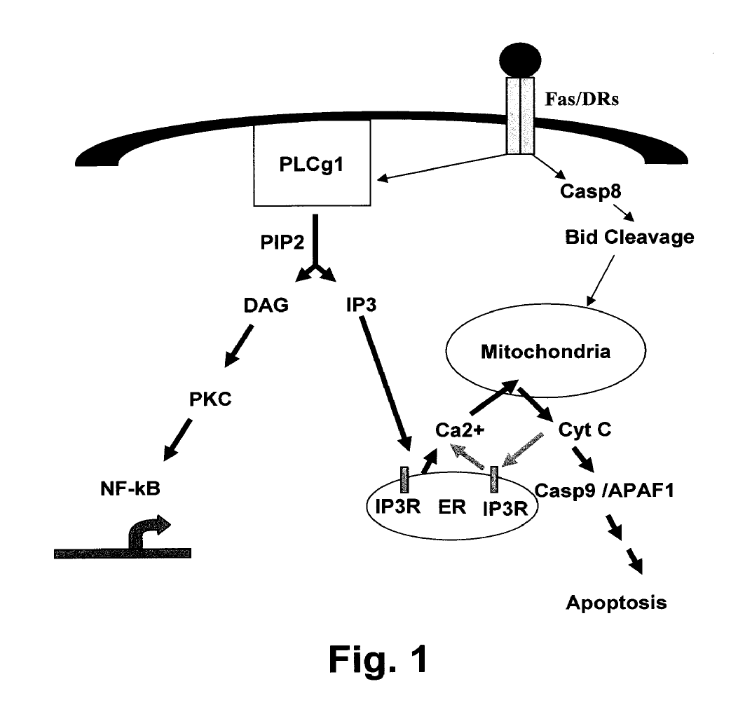

The calcium flux appears to have at least two components: an initial release of intracellular calcium from the ER and a later release of calcium triggered by cytochrome C. Cytochrome C is released from mitochondria due to increased permeability of the outer mitochondrial membrane, and serves a regulatory function as it precedes morphological change associated with apoptosis. Once cytochrome C is released it binds with Apaf-1 and ATP, which then bind to pro-caspase-9 to create a protein complex known as apoptosome. The apoptosome cleaves the pro-caspase to its active form caspase-9, which in turn activates other caspases and triggers a cascade of events leading to apoptosis. A proposed mechanism between calcium flux and apoptosis is shown in FIG. 1.

### HDAC Inhibitors

In eukaryotic cells, chromatin associates with histones to form nucleosomes. Each nucleosome consists of a protein octamer made up of two copies of each of histones H2A, H2B, H3 and H4. DNA winds around this protein core, with the basic amino acids of the histones interacting with the negatively charged phosphate groups of the DNA. The most common posttranslational modification of these core histones is the reversible acetylation of the ε-amino groups of the conserved, highly basic N-terminal lysine residues. Reversible acetylation of histones is a major regulator of gene expression. By altering the accessibility of transcription factors to DNA gene expression can be regulated.

In normal cells, histone deacetylases (HDAC) and histone acetyltransferases (HAT) control the level of acetylation of histones. Inhibition of HDAC results in the accumulation of hyperacetylated histones, which results in a variety of cellular responses.

Histone acetylation and deacetylation has long been linked to transcriptional control. In some embodiments, HDAC inhibitors, including, trichostatin A, sodium butyrate, suberoylanilide hydroxamic acid (SAHA), depsipeptide, MS-275, and aphicidin, among others, promote histone acetylation, resulting in relaxation of the chromatin structure. Chromatin relaxation and uncoiling permits and enhances the expression of diverse genes, including those involved in the differentiation process, e.g. p21^{CIP1}. In fact, HDAC inhibitors, for example SAHA, sodium butyrate, have been shown to induce maturation in various human leukemia cell lines.

Mammalian HDACs are divided into three major classes based on their structural or sequence homologies to the three distinct yeast HDACs: Rpd3 (class I), Hda1 (class II), and Sir2/Hst (class III). The Rpd3 homologous class I includes HDACs 1, 2, 3, 8, and 11; the Hda1 homologous class II includes HDACs 4, 5, 6, 7, 9 (9a and 9b), and 10; the Sir2/Hst homologous class III SIR T1, 2, 3, 4, 5, 6, and 7. Recent studies revealed an additional family of cellular factors that possesses intrinsic HAT or HDAC activities. These appear to be non-histone proteins that participate in regulation of the cell cycle, DNA repair, and transcription. A number of transcriptional coactivators, including but not limited to p400AF, BRCA2, and ATM-like proteins, function as HAT's. Some transcriptional repressors exhibit HDAC activities in the context of chromatin by recruiting a common chromatin-modifying complex. For instance, the Mas protein family (Mas1, Mxi1, Mad3, and Mad4) comprises a basic-helix-loop-helix-loop-helix-zipper class of transcriptional factors that heterodimerize with Max at their DNA binding sites. Mad:Max heterodimers act as transcriptional repressors at their DNA binding sites through recruitment of “repressor complexes.” Mutations that prevent interaction with either Max or the msin3 corepressor complex fail to arrest cell growth. Accordingly, HDAC inhibitor used herein refers to any agent capable of inhibiting the HDAC activity from any of the proteins described above.

Inhibitors of HDAC have been studied for their therapeutic effects on cancer cells. Butyric acid and its derivatives, including sodium phenylbutyrate, have been reported to induce apoptosis in vitro in human colon carcinoma, leukemia and retinoblastoma cell lines. Other inhibitors of HDAC that have been widely studied for their anti-cancer activities include trichostatin A and trapoxin.

### Pan-HDAC Inhibitors

By way of nonlimiting example, pan-HDAC inhibitors include short-chain fatty acids such as butyrate, 4-phenylbutyrate or valproic acid; hydroxamic acids such as suberoylanilide hydroxamic acid (SAHA), biaryl hydroxamate A-161906, bicyclic aryl-N-hydroxycarboxamides, CG-1521, PXD-101, sulfonamide hydroxamic acid, LAQ-824, oxamflatin, scriptaid, m-carboxy cinnamic acid bishydroxamic acid, trapoxin-hydroxamic acid analogue, trichostatin A, trichostatin C, m-carboxycinnamic acid bis-hydroxamideoxamflatin (CBHA), ABHA, Scriptaid, pyroxamide, and propenamides; epoxyketone-containing cyclic tetrapeptides such as trapoxins, apidicin, depsipeptide, HC-toxin, chlamydocin, diheteropeptin, WF-3161, Cy1-1 and Cy1-2; benzamides or non-epoxyketone-containing cyclic tetrapeptides such as FR901228, apicidin, cyclic-hydroxamic-acid-containing peptides (CHAPs), benzamides, MS-275 (MS-27-275), and CI-994; depudecin; PXD101; organosulfur compounds; and aroyl-pyrrolylhydroxy-amides (APHAs).

In some embodiments, the pan-HDAC inhibitor is PCI-24781, SAHA (Zolinza), trichostatin A, MS-275, LBH-589, PXD-101, MGCD-0103, JNJ-26481585, R306465 (J&J), or sodium butyrate.

In some embodiments, the pan-HDAC inhibitor is a compound selected from a compound or formula disclosed in U.S. patent application Ser. Nos. 10/818,755; 10/537,115; 10/922,119; 11/100,781; 11/779,743; PCT Patent Application No. PCT/US2005/046255 or U.S. Pat. No. 7,276,612; the disclosures of these references are herein incorporated in their entirety.

In some embodiments, the pan-HDAC inhibitor has the structure of Formula (I):

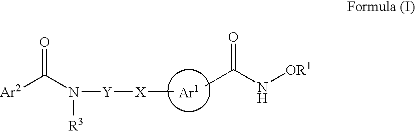

wherein:
R¹ is hydrogen or alkyl;
X is —O—, —NR²—, or —S(O)ₙ where n is 0-2 and R² is hydrogen or alkyl;
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl, halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl;
   and individual stereoisomers, individual geometric isomers, or mixtures thereof; or a pharmaceutically acceptable salt thereof.

### HDAC8 and HDAC8 Inhibitors

MACS is a 377 residue, 42 kDa protein localized to the nucleus of a wide array of tissues, as well as several human tumor cell lines. The wild-type form of full length HDAC8 is described in GenBank Accession Number NP 060956. The HDAC8 structure was solved with four different hydroxamate inhibitors bound.

HDAC8 is an HDAC isoform with deacetylase activity in vitro that is expressed in multiple tissue types and tumor cell lines. Based on sequence homology, HDAC8 is considered to be a Class I enzyme, although phylogenetic analysis has shown it to lie near the boundary of the Class I and Class II enzymes. HDAC8 is different from the prototypical Class I enzyme in several respects, including its reported cytoplasmic—as opposed to nuclear—subcellular localization, the binding of various metals including Fe(II) and K+ to its active site, and the negative regulation of its catalytic activity by phosphorylation of Ser39 by cyclic-AMP dependent protein kinase (PKA)20-22. The three dimensional crystal structure of human HDAC8 was recently solved and revealed unique features of HDAC8, including conformational flexibility proximal to the binding site pocket mediated by the L1 active site loop, and a unique influence of Ser39 phosphorylation on active site inhibition.

HDAC8 is expressed primarily in delta cells of the islets of Langerhans in the pancreas; in small intestinal epithelial cells; and in neuroendocrine cells. Of note, delta cells express and secrete somatostatin, a peptide hormone that inhibits the secretion of insulin and growth hormone. Without being bound by theory, it is believed that HDAC8 activity drives the expression of somatostatin in delta cells. Thus, inhibiting HDAC8 activity is expected to decrease somatostatin expression and secretion from delta cells and, consequently, increase systemic insulin and growth hormone levels.

HDAC8 is expressed at high levels in tumor cell lines. Examples include the lines: Jurkat, HuT78, K562, PC3, and OVCR-3. It has been demonstrated that inhibiting HDAC8 activity decreases the proliferation of T-cell derived tumor cells (e.g., Jurkat cells) by induction of apoptosis. HDAC8 inhibition does not affect the proliferation of either non-cancerous cells or tumor cell lines other than T-cell derived lines. Thus, selective HDAC8 inhibitors are useful for slowing or arresting the progression of T-cell derived cancers while demonstrating lessened or no toxicity to non-cancerous cells.

Selective HDAC8 inhibitor compounds and compositions thereof are used, in certain embodiments, to treat a subject suffering from a T-cell lymphoma, e.g., a peripheral T-cell lymphoma, a lymphoblastic lymphoma, a cutaneous T-cell lymphoma, or an adult T-cell lymphoma.

In some embodiments, the HDAC8 inhibitor is selected from the group consisting of: indole-6-carboxylic acid hydroxyamide compounds and indole-5-carboxylic acid hydroxyamide compounds, indole derivatives, pharmaceutically acceptable salts thereof, pharmaceutically acceptable N-oxides thereof, pharmaceutically active metabolites thereof, pharmaceutically acceptable prodrugs thereof, and pharmaceutically acceptable solvates thereof.

In some embodiments, the HDAC8 inhibitor is selected from the group consisting of: PCI-34051, PCI-46646, PCI-34260, PCI-34263, R306465 (J&J), and derivatives thereof.

In some embodiments, the HDAC8 inhibitor is a compound selected from a compound or formula disclosed in U.S. Patent Application No. 60/911,857; 60/944,409; 60/954,777; 11/779,743; 60/865,825; 11/940,232; 11/687,565; or PCT Patent Application No. PCT/US2007/073802; PCT/US2007/084718; PCT/UC2007/06714; the disclosures of these references are herein incorporated in their entirety.

In some embodiments, the HDAC8 inhibitor is a hydroxamic acid having the structure of Formula (A):

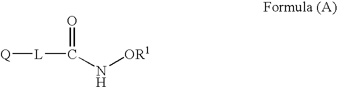

wherein:

Q is an optionally substituted C₅₋₁₂ aryl or an optionally substituted C₅₋₁₂ heteroaryl;

L is a linker having at least 4 atoms;

R¹ is H or alkyl;

and a pharmaceutically acceptable salt, pharmaceutically acceptable N-oxide, pharmaceutically active metabolite, pharmaceutically acceptable prodrug, pharmaceutically acceptable solvate thereof.

### Calcium Flux as a Rapid Pharmacoefficacy Biomarker for HDAC Inhibitors

Experiments have demonstrated that calcium flux correlates with a cell's ability to undergo apoptosis. Positive calcium flux will often preceed apoptosis while lack of, or negative, calcium flux usually indicates the cell will not undergo apoptosis. Thus, in some embodiments, the level of calcium flux is measured in order to determine if a patient will respond to treatment with an apoptotic agent. In some embodiments, the level of calcium flux is measured in order to determine if a patient should be included in a clinical trial of an apoptotic agent.

In some embodiments, the calcium flux correlates to a % level of apoptosis of at least 10% or more. In some embodiments, the calcium flux correlates to a % level of apoptosis of at least 15% or more. In some embodiments, the calcium flux correlates to a % level of apoptosis of at least 20% or more. In some embodiments, the calcium flux correlates to a % level of apoptosis of at least 25% or more. In some embodiments, the calcium flux correlates to a % level of apoptosis of at least 30% or more.

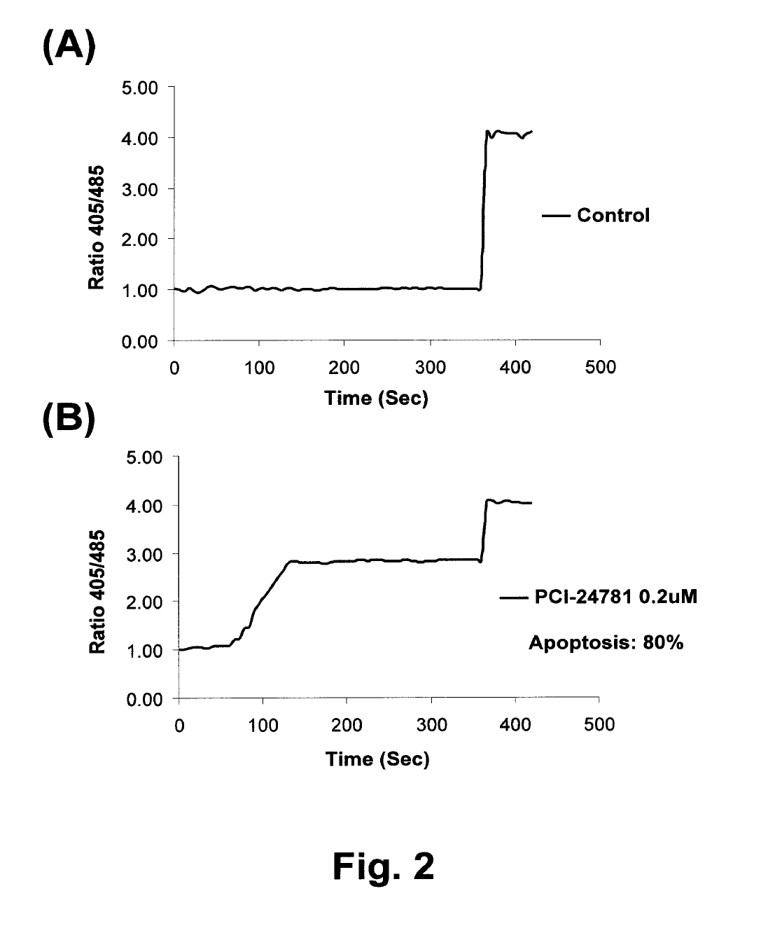

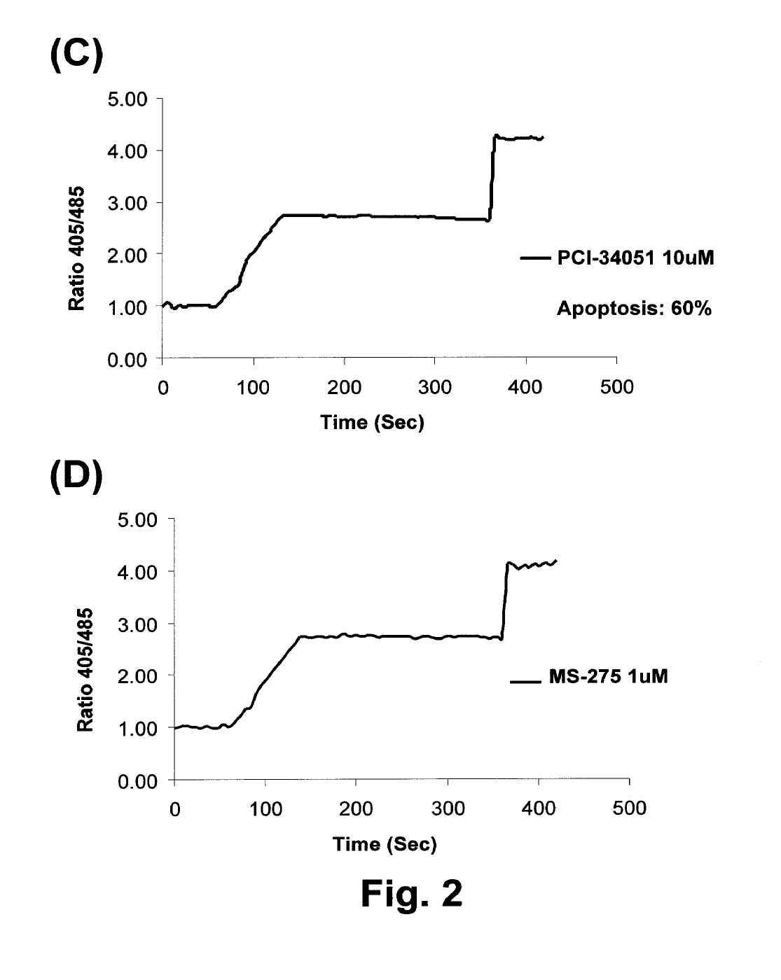

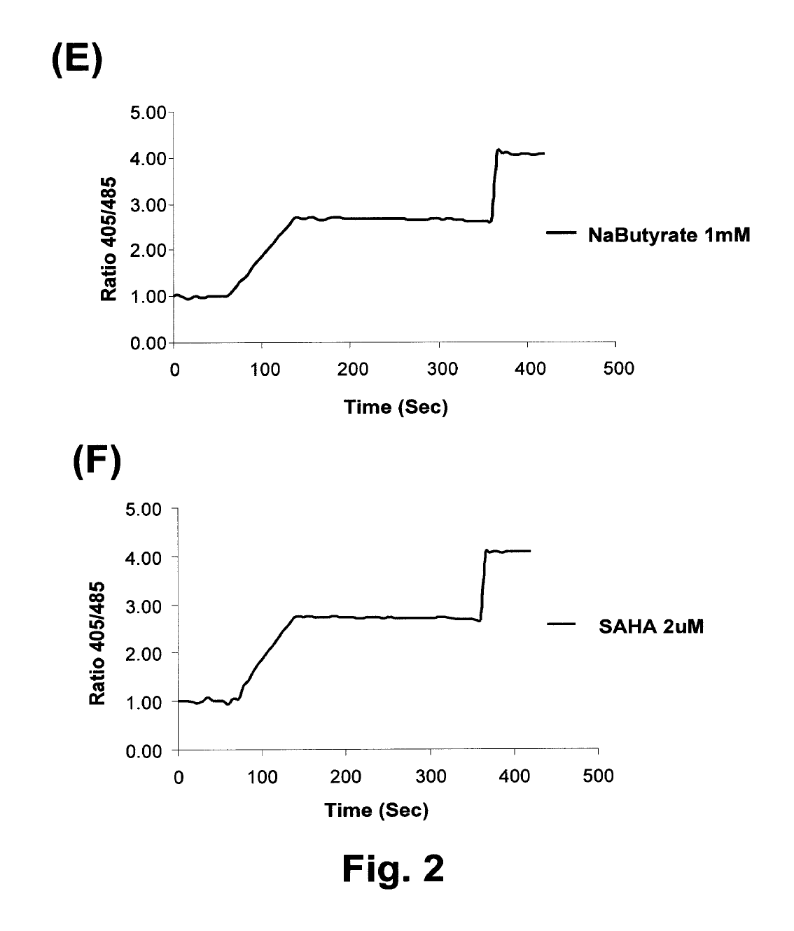

FIG. 2 shows the correlation between calcium flux and apoptosis in Jurkat cells. Jurkat cells were treated with the pan-HDAC Inhibitor, PCI-24781, (0.2 μM). After addition of PCI-24781 a measurable increase in intracellular calcium was observed. Further, after 2-day treatments with PCI-24781 the percentage of apoptosis was measured by Annexin-V. The total amount of apoptosis was 80%. Jurkat cells were also treated with the HDAC8 inhibitor PCI-34051 (10 μM). After addition of this HDAC8 inhibitor, a measurable increase in intracellular calcium was observed. After a further 2 days of treatment with PCI-34051, the levels of apoptosis were determined by Annexin-V. The percentage of apoptosis was 60%. Experiments were also performed with 1 μM MS-275; 1 mM NaButyrate; and 2 μM SAHA. In all cases, a positive calcium flux response was followed by a high percentage of cells undergoing apoptosis.

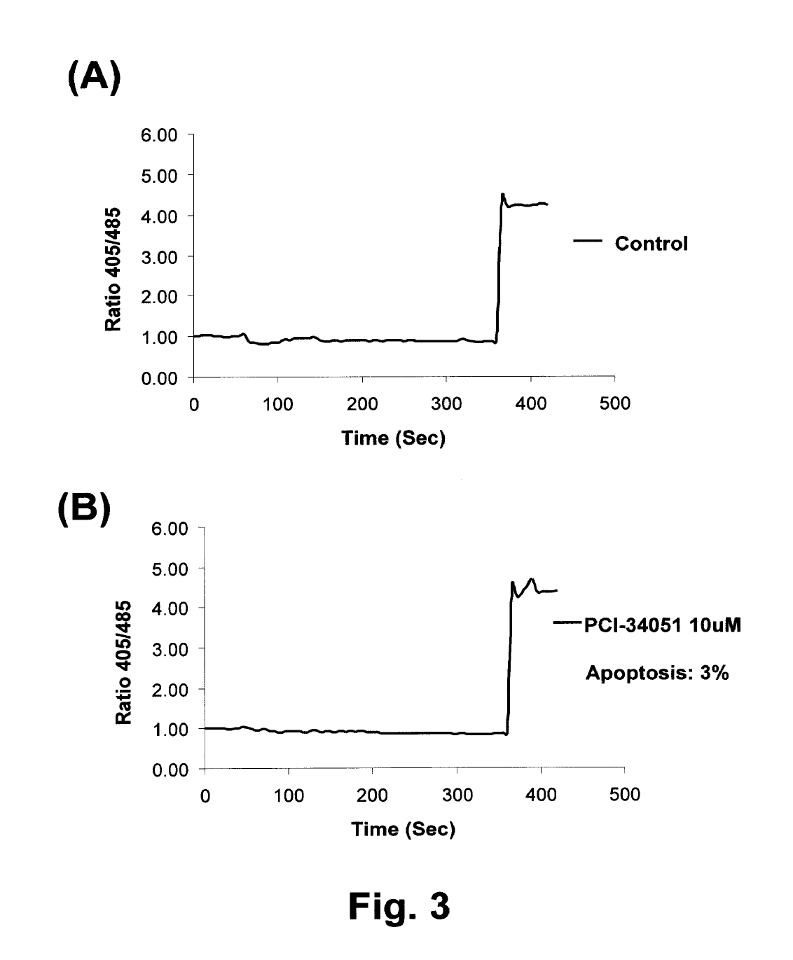

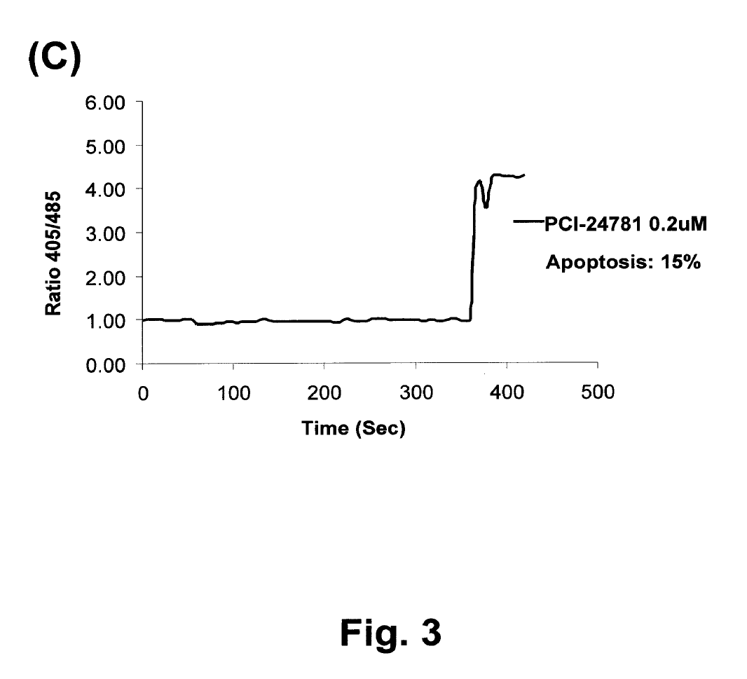

FIG. 3 shows calcium flux dependency on the enzyme PLCγ. Tumor cell lines lacking PLCγ (human hepatocarcinoma (HH) cells: T-cell lymphoma) were treated with the pan-HDAC inhibitor PCI-24781 and the HDAC8 inhibitor PCI-34051. Neither HDAC inhibitor induced calcium flux. Also shown are the percentages of apoptosis as measured by Annexin-V after 2-day treatments with PCI-24781 and PCI-34051. (A) Control; (B) 10 μM HDAC8 inhibitor PCI-34051; (C) 0.2 μM pan-HDAC inhibitor PCI-24781. It is theorized that a pan-HDAC inhibitor or an HDAC8 inhibitor activates PLCγ, thus triggering the calcium-mediated downstream pathway leading to apoptosis. These experiments also demonstrate that calcium flux is linked to apoptosis: PLCγ mutant cells (HH cells) did not undergo apoptosis.

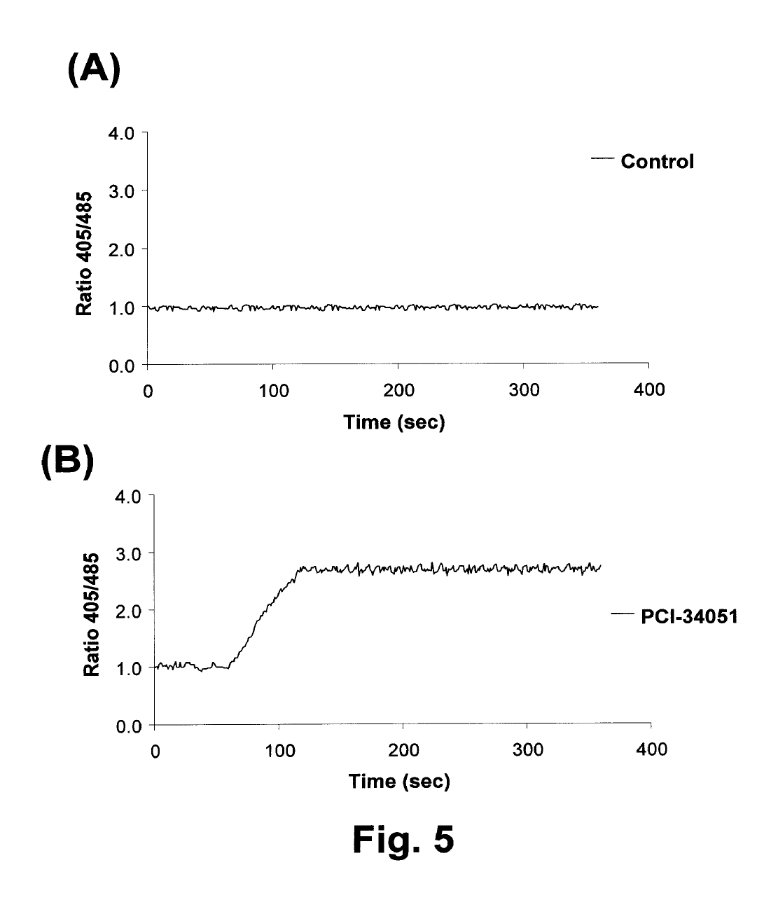

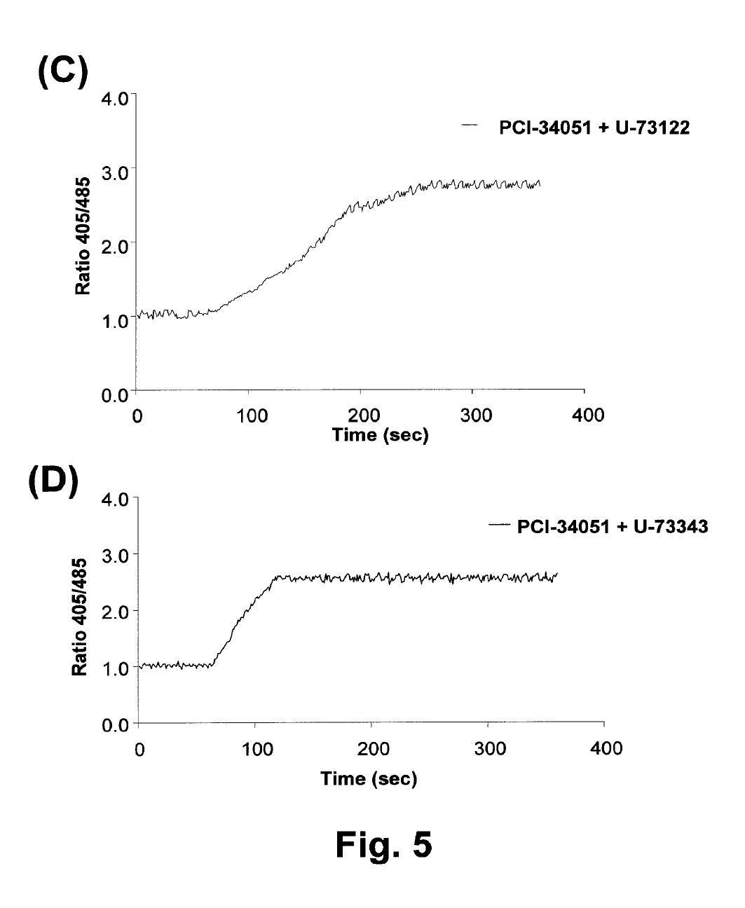

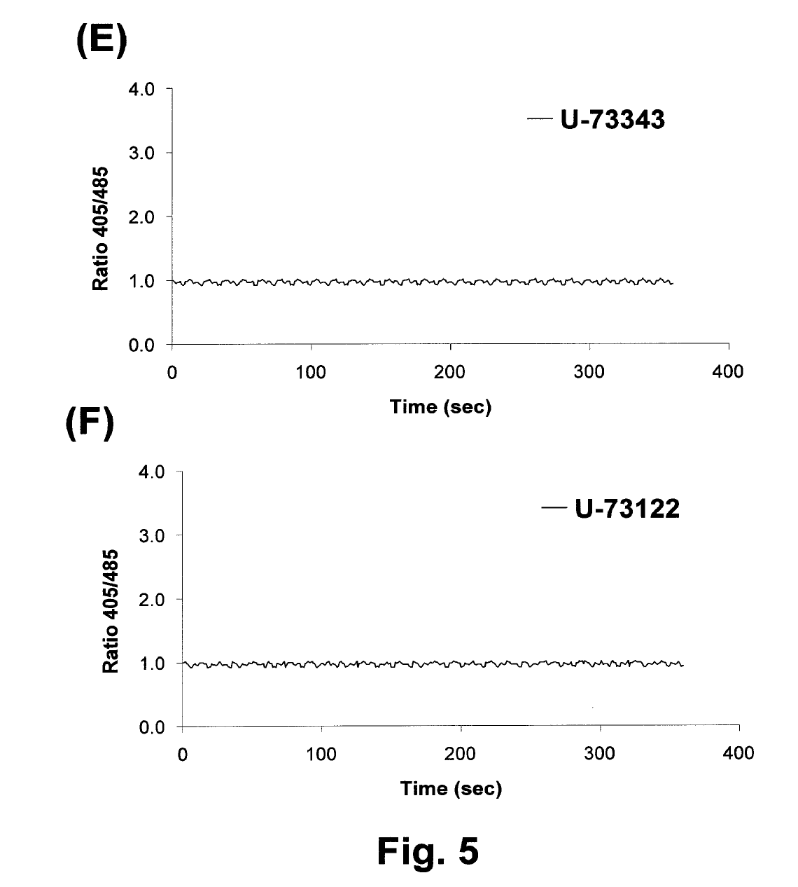

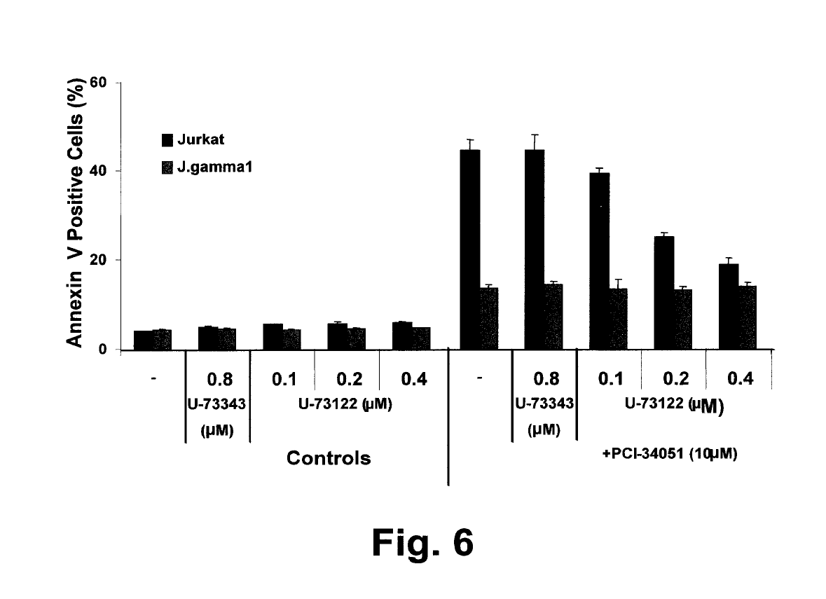

FIGS. 5 and 6 further demonstrate the dependency of calcium flux on PLCγ. Tumor cell lines containing the enzyme PLCγ (Jurkat cells) were treated with inhibitors of PLCγ (inhibitor U-73122: 1-[6-((17b-3-Methoxyestra-1,3,5(10)-trien-17-yl)amino)hexyl]-1H-pyrrole-2,5-dione; inhibitor U-73343: 1-[6-((17b-3-Methoxyestra-1,3,5(10)-trien-17-yl)amino)hexyl]-2,5-pyrrolidinedione) alone or in combination with the HDAC8 inhibitor PCI-34051. Tumor cell lines containing the enzyme PLCγ (Jurkat cells) but treated with a PLCγ inhibitor did not undergo calcium flux. (FIG. 5: (E) and (F)). However, the same Jurkat cells treated with both an HDAC8 inhibitor and a PLCγ inhibitor did calcium flux or had a delayed calcium flux. (FIG. 5: (B), (C), and (D)). These experiments also demonstrate that calcium flux is linked to apoptosis: blocking the calcium flux with PLCγ inhibitors (U-73343 and U-73122) blocked apoptosis (FIG. 6).

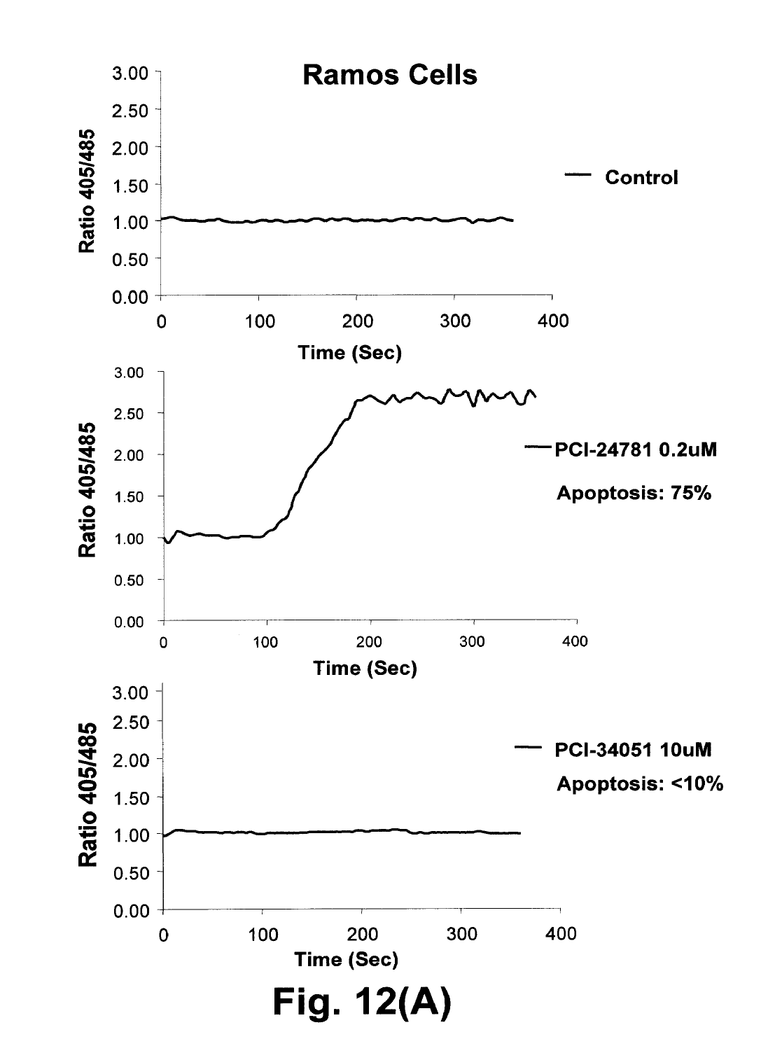

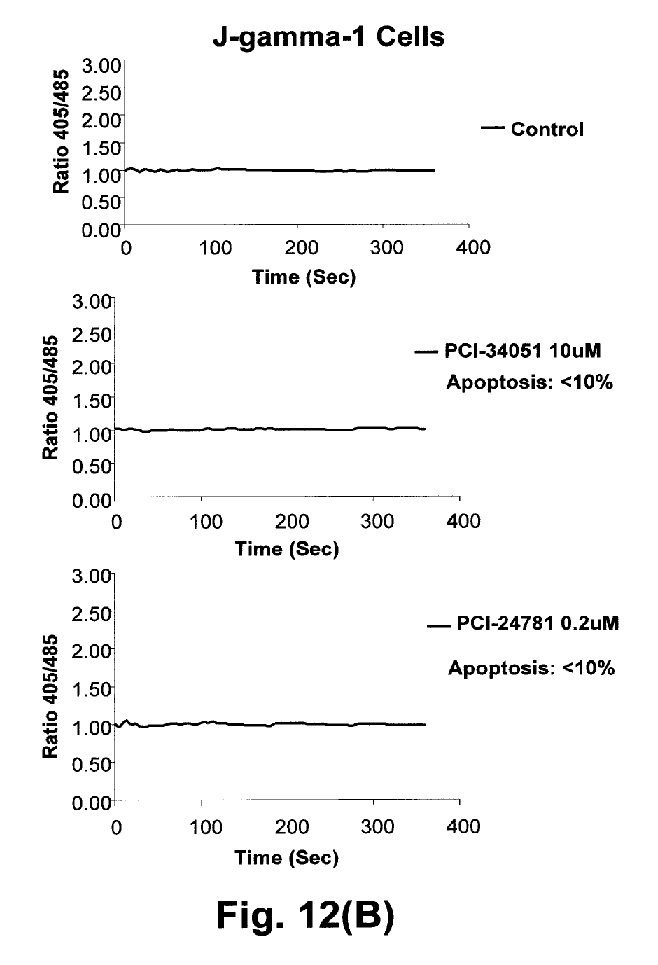

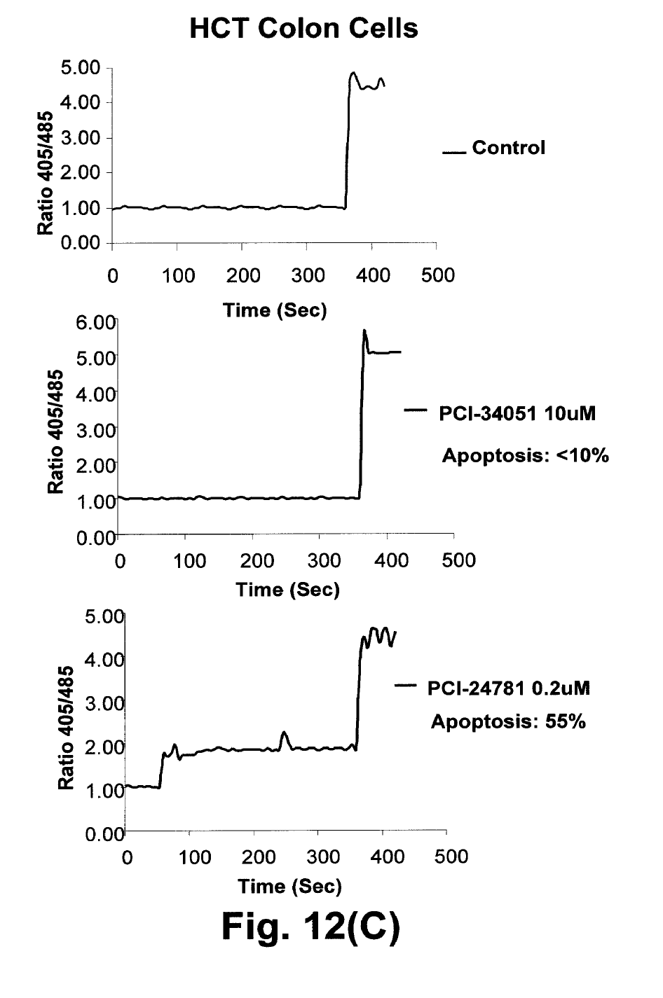

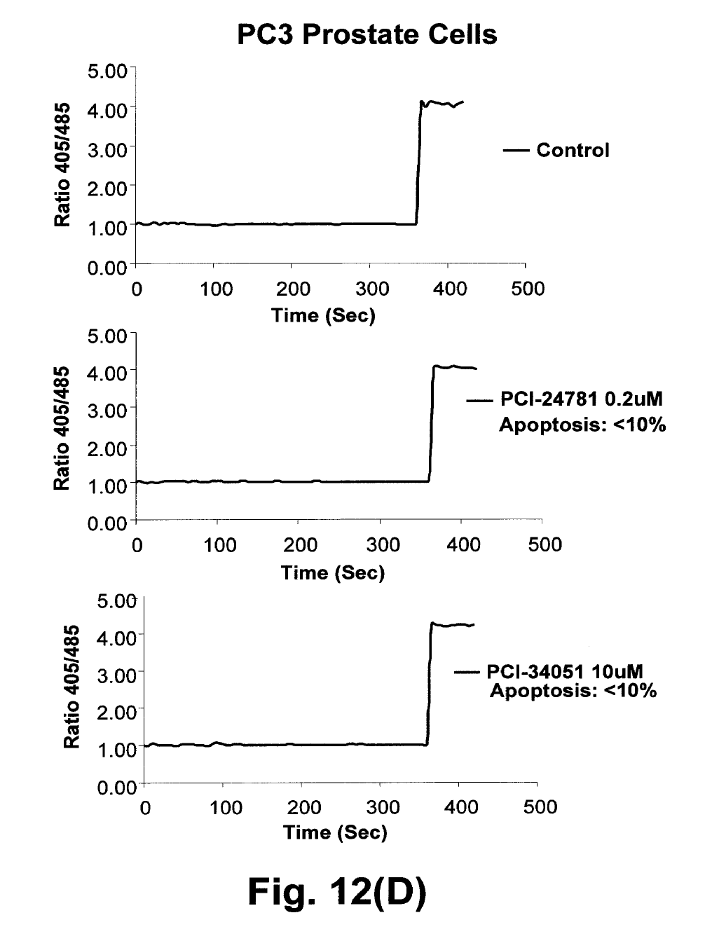

FIG. 12 demonstrates that calcium flux can be used to predict sensitivity to a pan-HDAC inhibitor or an HDAC8 inhibitor. FIG. 12(A) shows induction of calcium flux by PCI-24781, but not PCI-34051 in Ramos cells. (B) shows no induction of calcium flux by either PCI-24781 or PCI-34051 in J.gamma1 cells. (C) shows induction of calcium flux by PCI-24781 but not PCI-34051 in HCT-116 colon cells. (D) shows no induction of calcium flux by either PCI-24781 or PCI-34051 in PC3 prostate cells. Tumor cell lines that underwent apoptosis following treatment with PCI-24781 or PCI-34051 experienced a calcium flux. Tumor cell lines that did not undergo apoptosis following treatment with PCI-24781 or PCI-34051 did not experience a calcium flux.

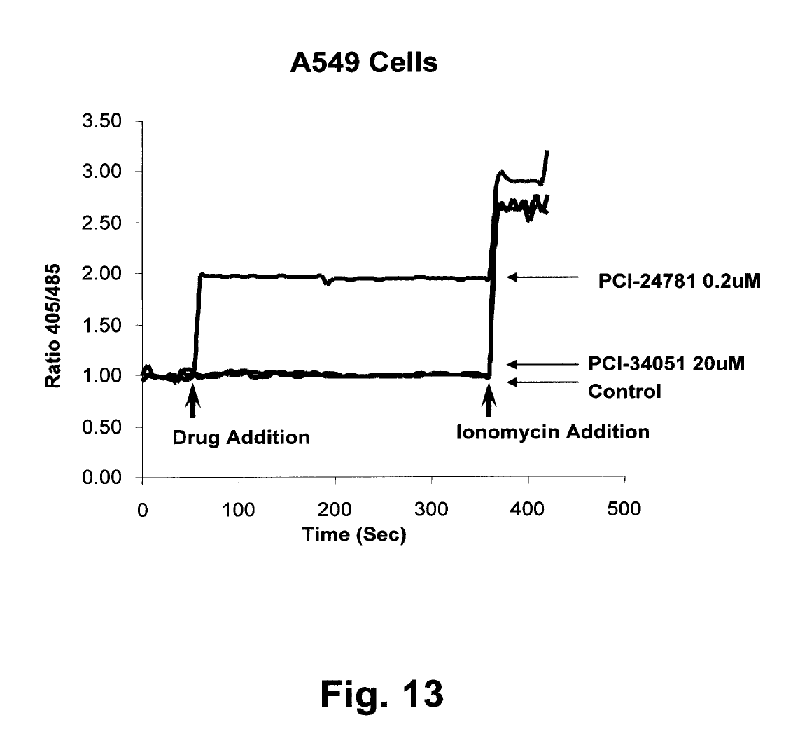

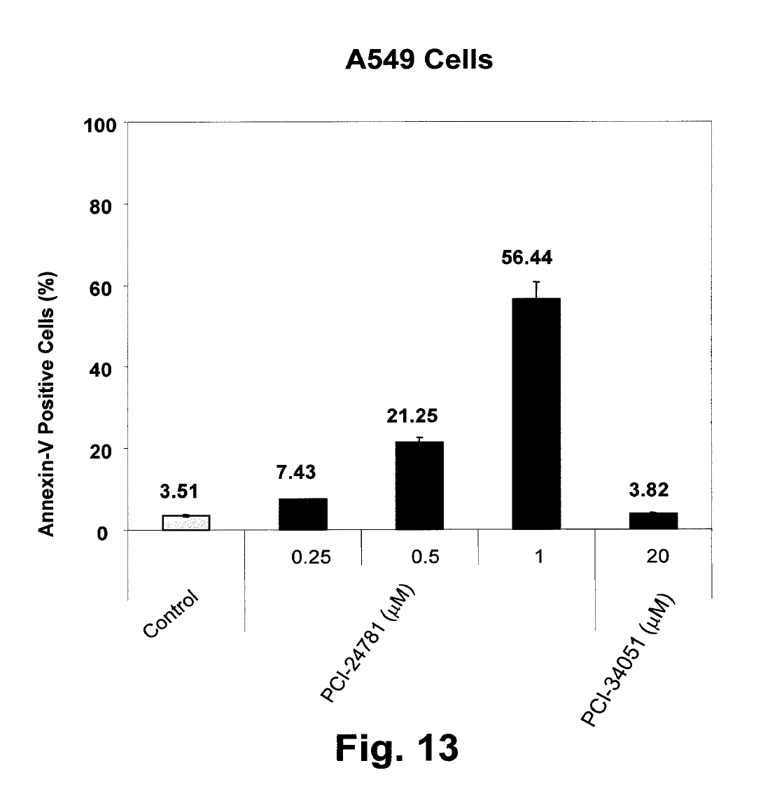

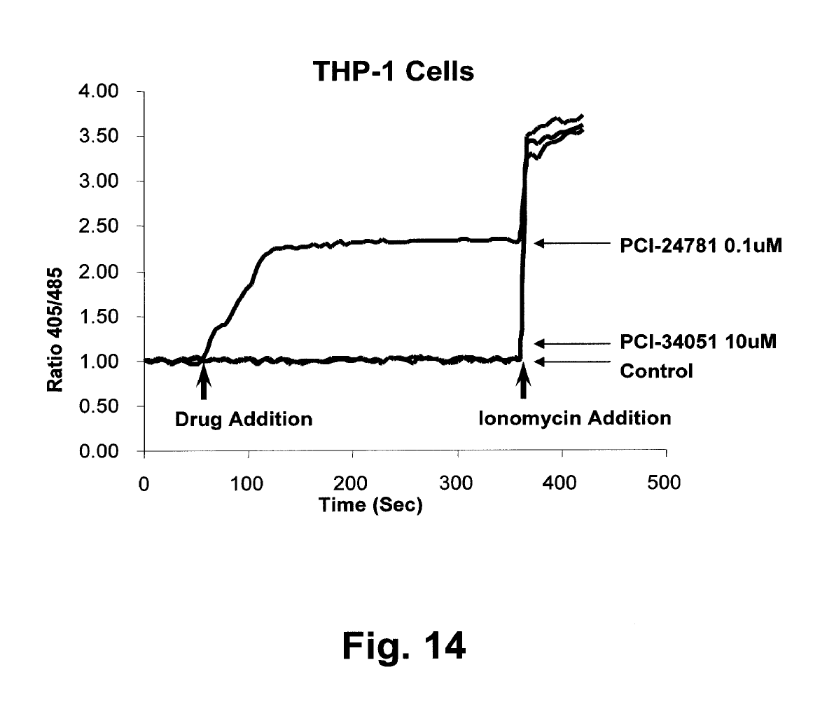

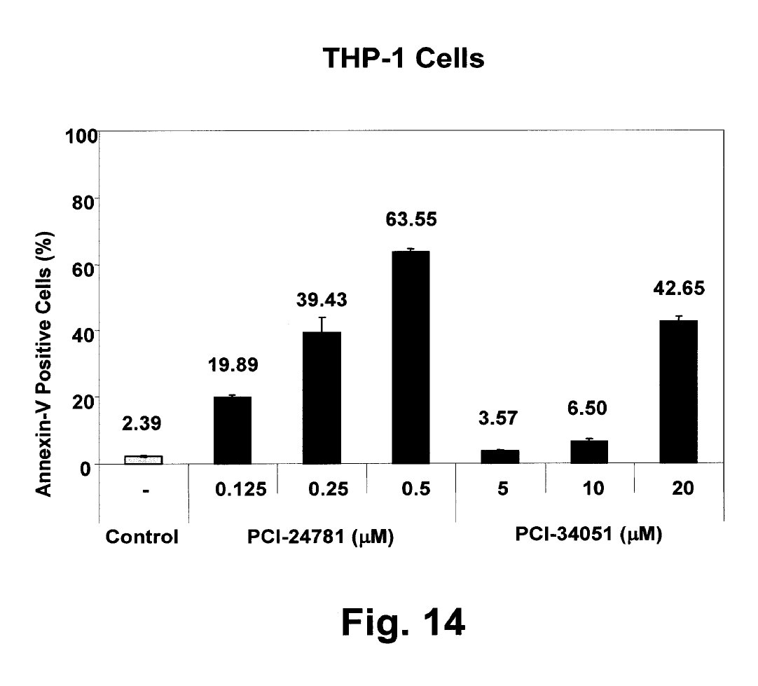

FIGS. 13 and 14 reflect assays performed in order to correlate sensitivity to the pan-HDAC inhibitor, PCI-24781, and the HDAC8 inhibitor, PCI-34051, with the ability to undergo calcium flux. Two tumor cell lines (A549 lung tumor, THP-1 monocytic leukemia) were tested. FIG. 13 shows induction of calcium flux by PCI-24781, but not PCI-34051 in A549 cells. The A549 undergo apoptosis in response to PCI-24781. This apoptosis was predicted by a calcium flux. The T-cell specific HDAC8 selective inhibitor PCI-34051 neither induces apoptosis nor calcium flux in these cells. FIG. 14 shows induction of calcium flux by PCI-24781, but not PCI-34051 in THP-1 cells. THP-1 is a monocytic leukemia line in which PCI-24781 induces apoptosis and calcium flux, and PCI-34051 does not.

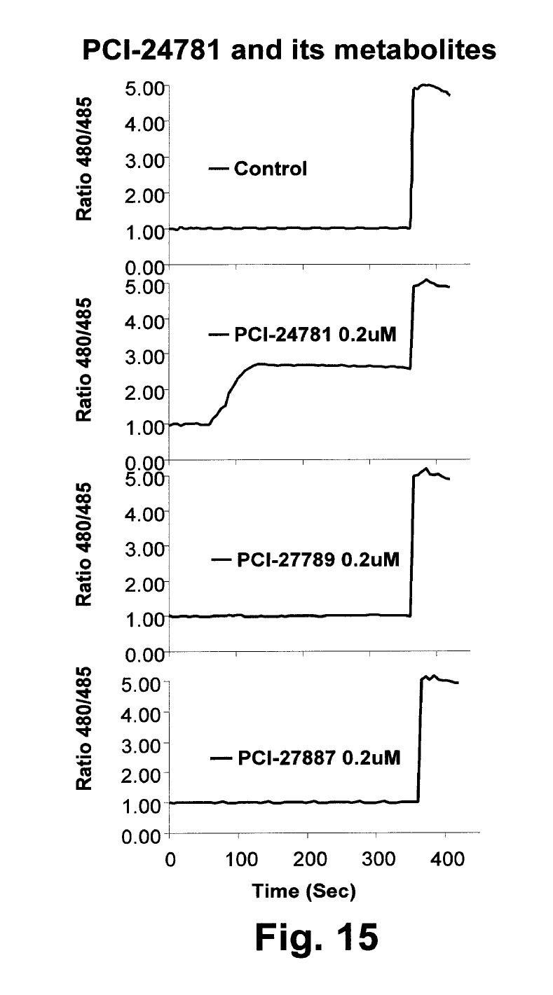

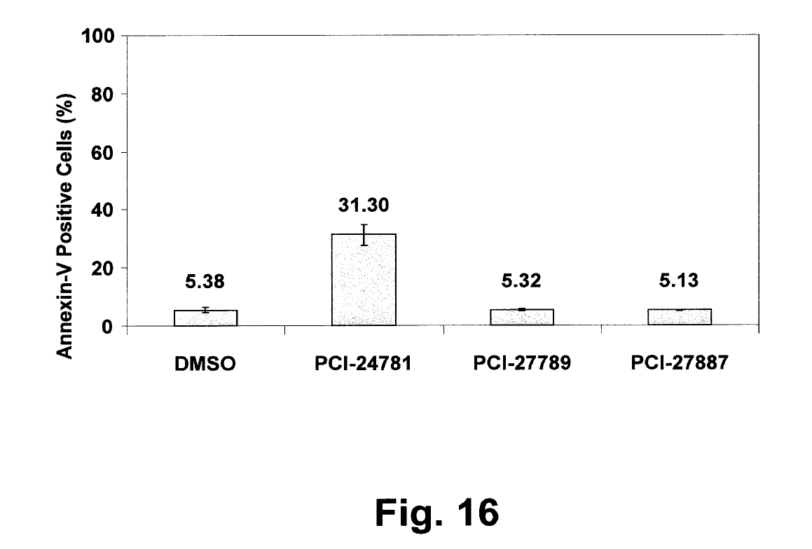

FIGS. 15 and 16 reflect assays performed in order to correlate sensitivity of the pan-HDAC inhibitor, PCI-24781, and two major metabolites of PCI-24781 (PCI-27789: carboxylic acid metabolite and PCI-27787: amide metabolite) with the ability to undergo calcium flux. Jurkat cells were tested. FIG. 15 shows rapid induction of calcium mobilization in Jurkat cells by PCI-24781. Two metabolites of PCI-24781, the carboxylic acid metabolite PCI-27789 and the amide metabolite PCI-27787, have no histone deacetylase

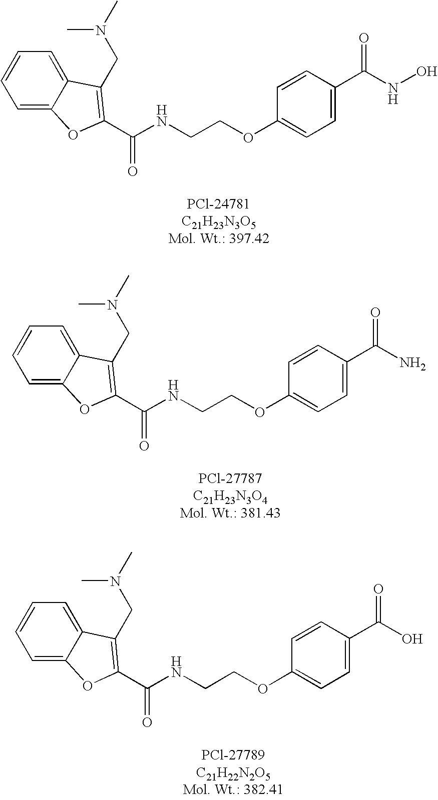

(HDAC) activity and were not able to cause induction of calcium flux in the Jurkat cells. FIG. 16 shows induction of apoptosis by PCI-24781, while PCI-27789 and PCI-27787 have no apoptosis-inducing effect in Jurkat cells.

The chemical structures of the two metabolites are similar to PCI-24781. They only differ from PCI-24781 in the “active” portion of the molecule: PCI-27789 has a carboxylic acid group instead of the hydroxamic acid group of PCI-24781; PCI-27787 has an amide group instead of the hydroxamic acid group of PCI-24781.

In some embodiments, calcium flux has utility as a clinical biomarker that aids in determining whether a tumor is sensitive to an apoptotic agent. Thus, in some embodiments, calcium flux is used to select and predict patients likely to respond to a pan-HDAC inhibitor or an HDAC8 inhibitor.

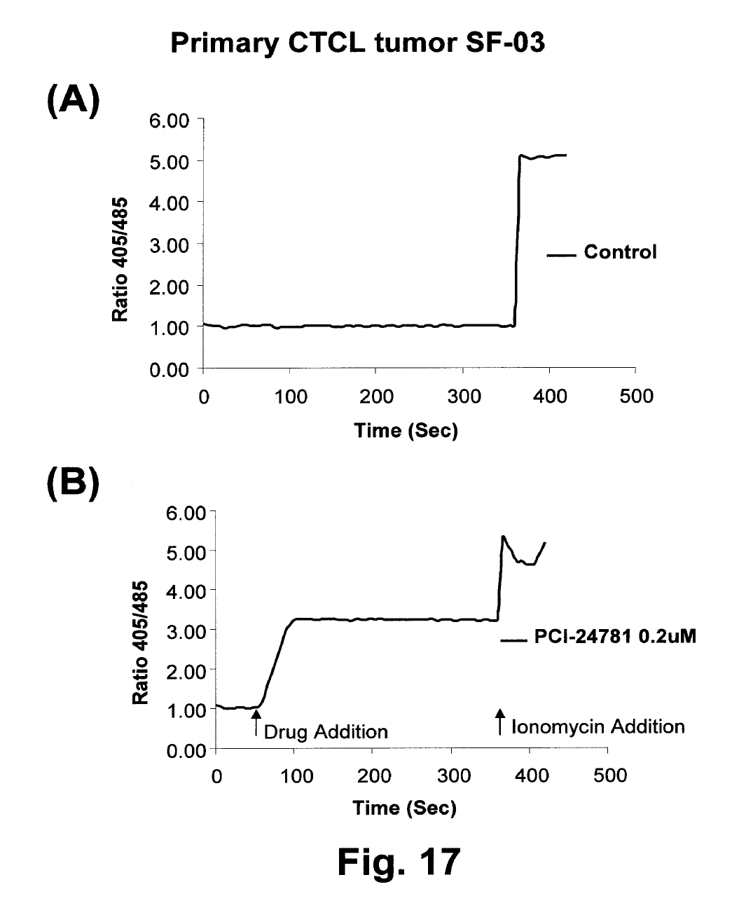

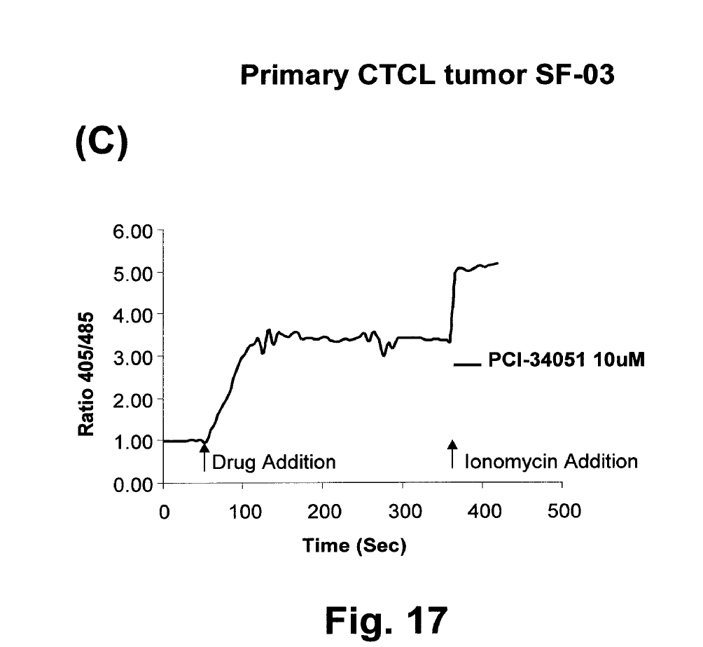

FIG. 17 shows a positive calcium flux measured in a primary tumor biopsy from a patient with Cutaneous T-cell lymphoma (CTCL) (patient SF-03). The cells derived from the 5 mm punch biopsy were treated with collagenase, and incubated in growth media to which was added the pan-HDAC inhibitor, PCI-24781, or the HDAC8 inhibitor, PCI-34051. Both compounds led to a rapid and statically significant calcium flux within 5 seconds of their coming into contact with the cells. Both compounds also led to an increase in tumor cell apoptosis following 40 hours of treatment.

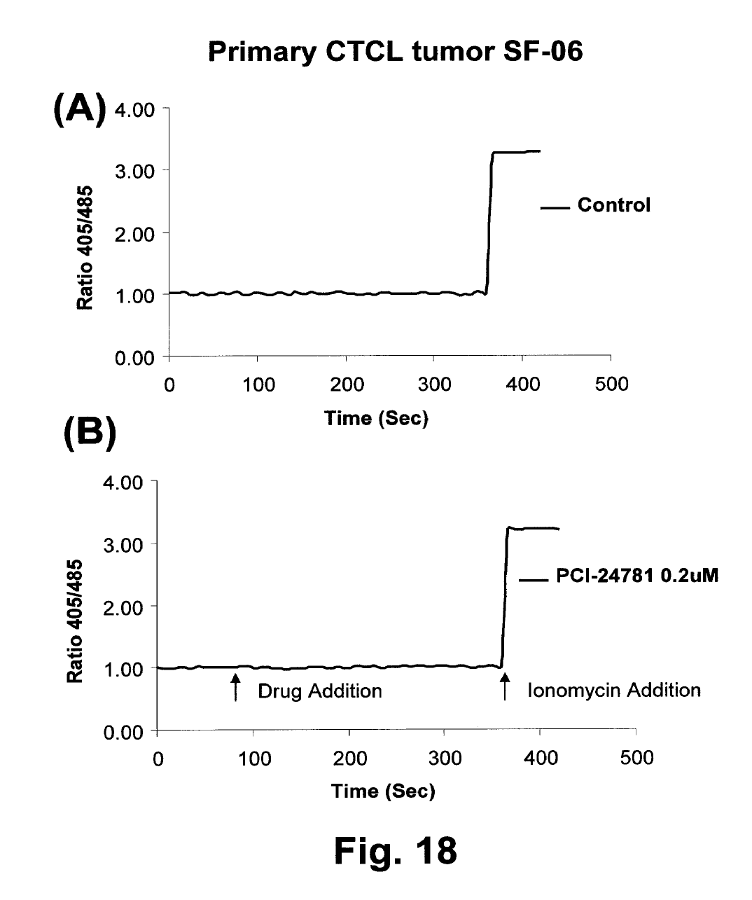

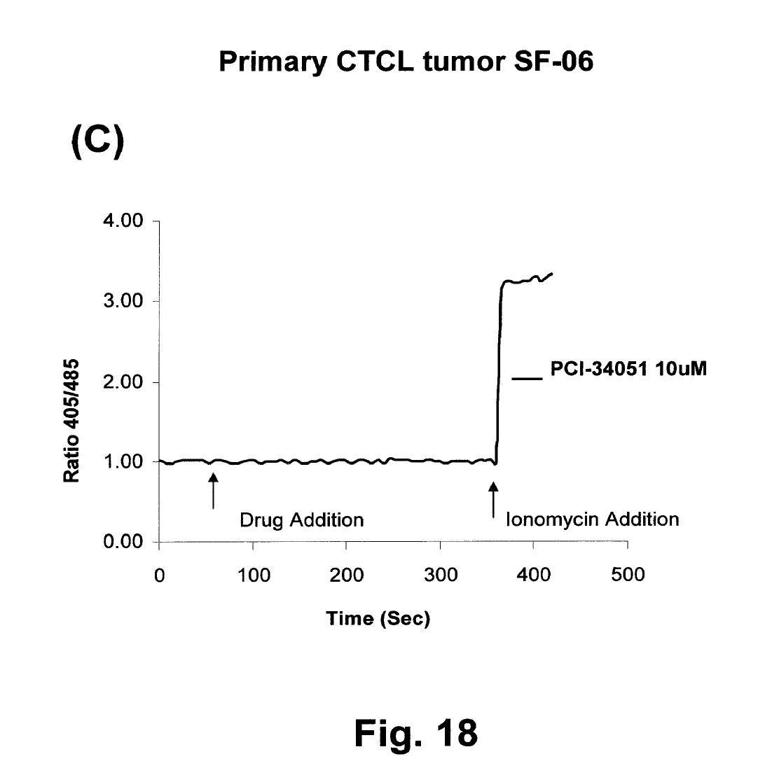

FIG. 18 shows a negative calcium flux measured in a primary tumor biopsy from a second CTCL patient (patient SF-06). The cells derived from the 5 mm punch biopsy were treated with collagenase, and incubated in growth media to which was added the pan-HDAC inhibitor, PCI-24781, or the HDAC8 inhibitor, PCI-34051. Neither HDAC inhibitor stimulated calcium flux nor lead to apoptosis of the tumor cells.

Calcium Flux Assay

Cell Extraction from Primary Tumors

Tumor biopsies are obtained from a prospective subject. The biopsies are placed into RPMI 1640 in a 50 ml Falcon tube immediately after excision and stored on ice. They are shipped to the processing site as soon as possible. The supernatant that the tumor sample came in is saved. The tumor is minced into fragments <0.3-0.5 mm thick, and digested with 10 ml RPMI 1640 containing 1 mg/ml collagenase D (Roche) for 30 min at 37° C. on a shaker. The digestion is stopped by adding 10 mM EDTA, and putting it on ice. The digested tissue is washed 3× with ice cold PBS/10 mM EDTA 3X, and is then combined with the retained supernatant. The digested tissue and supernatant are passed through a 70-μm pore nylon mesh (BD Biosciences) and centrifuged (300 g) for 20 min at 4° C. The liquid is poured off leaving extracted cells. The extracted cells are resuspended in RPMI complete medium to a concentration of 1×10⁶ cells/mL. For drug treatment and assays, the cells are cultured for about 2 days in RPMI+10% FBS in a 37° C. incubator.

Circulating Tumor Cell Extraction from Blood

Blood samples are obtained from a prospective subject. The RBCs are lysed with a red cell lysis buffer. Immunogenic beads coated with an epithelial specific antibody are applied according to the manufacturer's instructions. The beads are then washed several times to remove any non-epithelial cells. The extracted epithelial cells are resuspended in RPMI complete medium to a concentration of 1×10⁶ cells/mL. For drug treatment and assays, the cells are cultured for about 2 days in RPMI+10% FBS in a 37° C. incubator.

### Intracellular Calcium Measurements

To obtain the calcium flux levels before the apoptotic agent was added, an aliquot of cultured cells (1×10⁶ cells/mL) were incubated in the absence of light for 1 h in Hanks' Balanced Salt Solution (HBSS; Invitrogen) containing 10% Fetal Bovine Serum and 5 M Indo-1 AM (Invitrogen) at 37° C. Cells were then harvested, centrifuged (200×g for 5 min) and washed three times with HBSS to remove extracellular Indo-1, and readjusted to 1×10⁶ cells/mL in HBSS.

Fluorescence was monitored at 37° C. with a fluorescent plate reader (Fluoroskan Ascent FL; Thermo Scientific). After a 5 min temperature equilibrium period the samples were excited at 338 nm and emission was collected at 405 and 485 nm in kinetic mode at 6-sec intervals over a 5 min period. Maximal ratio values were determined by the addition of 10 μM ionomycin at the end of the measurements.

The apoptotic agent was added to a second aliquot of cultured cells (1×10⁶ cells/mL). The cells were incubated in the absence of light for 1 h in Hanks' Balanced Salt Solution (HESS; Invitrogen) containing 10% Fetal Bovine Serum and 5 μM Indo-1 AM (Invitrogen) at 37° C. Cells were then harvested, centrifuged (200×g for 5 min) and washed three times with HBSS to remove extracellular Indo-1 AM, and readjusted to 1×10⁶ cells/mL in HBSS. The cells were then incubated with the apoptotic agent for 5 minutes.

Fluorescence was monitored throughout each experiment at 37° C. with a fluorescent plate reader (Fluoroskan Ascent FL; Thermo Scientific). After a 5 min temperature equilibrium period the samples were excited at 338 nm and emission was collected at 405 and 485 nm in kinetic mode at 6-sec intervals over a 5 min period. Maximal ratio values were determined by the addition of 10 μM ionomycin at the end of the measurements. Intracellular [Ca²⁺] changes, or calcium flux, are shown as changes in the ratio of Indo-1 bound to free calcium (405 nm/485 nm).

### EXAMPLES

The following examples are to be construed as merely illustrative, and not limitative of the remainder of the disclosure in any way whatsoever.

Protocol for Extracting T-Cells from Tumor Tissue and Measuring Calcium Flux

### Example 1

### Intracellular Calcium Measurements

To obtain the calcium flux levels before the apoptotic agent was added, an aliquot of cultured cells (1×10⁶ cells/mL) were incubated in the absence of light for 1 h in Hanks' Balanced Salt Solution (HBSS; Invitrogen) containing 10% Fetal Bovine Serum and 5 M Indo-1 AM (Invitrogen) at 37° C. Cells were then harvested, centrifuged (200×g for 5 min) and washed three times with HBSS to remove extracellular Indo-1, and readjusted to 1×10⁶ cells/mL in HBSS.

Fluorescence was monitored at 37° C. with a fluorescent plate reader (Fluoroskan Ascent FL; Thermo Scientific). After a 5 min temperature equilibrium period the samples were excited at 338 nm and emission was collected at 405 and 485 nm in kinetic mode at 6-sec intervals over a 5 min period. Maximal ratio values were determined by the addition of 10 μM ionomycin at the end of the measurements.

The apoptotic agent was added to a second aliquot of cultured cells (1×10⁶ cells/mL). The cells were incubated in the absence of light for 1 h in Hanks' Balanced Salt Solution (HBSS; Invitrogen) containing 10% Fetal Bovine Serum and 5 μM Indo-1 AM (Invitrogen) at 37° C. Cells were then harvested, centrifuged (200×g for 5 min) and washed three times with HBSS to remove extracellular Indo-1 AM, and readjusted to 1×10⁶ cells/mL in HBSS. The cells were then incubated with the apoptotic agent for 5 minutes.

Fluorescence was monitored throughout each experiment at 37° C. with a fluorescent plate reader (Fluoroskan Ascent FL; Thermo Scientific). After a 5 min temperature equilibrium period the samples were excited at 338 nm and emission was collected at 405 and 485 nm in kinetic mode at 6-sec intervals over a 5 min period. Maximal ratio values were determined by the addition of 10 μM ionomycin at the end of the measurements. Intracellular [Ca²⁺] changes, or calcium flux, are shown as changes in the ratio of Indo-1 bound to free calcium (405 nm/485 nm).

### Example 2

### Apoptosis Measurements

Cytoxicity was evaluated after 2 or 3 days of treatment with and HDAC inhibitor and in combination with qVD, BAPTA-AM, thapsigargin and/or phospholipase C inhibitor using annexin-V staining. Annexin-V binding was assayed with a FACSCalibur instrument (Becton-Dickinson) using reagents from BioVision per manufacturer's protocol. See FIGS. 2, 3, 4, 6, 7, 12, and 16.

### Example 3

### Cell Extraction from Primary Tumors

Tumor biopsies are placed into RPMI 1640 in a 50 ml Falcon tube immediately after excision and stored on ice. They are shipped to the processing site as soon as possible. The supernatant that the tumor sample came in is saved. The tumor is minced into fragments <0.3-0.5 mm thick, and digested with 10 ml RPMI 1640 containing 1 mg/ml collagenase D (Roche) for 30 min at 37° C. on a shaker. The digestion is stopped by adding 10 mM EDTA, and putting it on ice. The digested tissue is washed 3× with ice cold PBS/10 mM EDTA 3X, and is then combined with the retained supernatant. The digested tissue and supernatant are passed through a 70-μm pore nylon mesh (BD Biosciences) and centrifuged (300 g) for 20 min at 4° C. The extracted cells are resuspended in RPMI complete medium to a concentration of 1×10⁶ cells/mL. For drug treatment and assays, the cells are cultured for about 2 days in RPMI+10% FBS in a 37° C. incubator.

### Example 4

### HDAC Inhibitor Compound-Induced Apoptosis Requires Phospholipase C-γ1 (PLC-γ1) Signaling

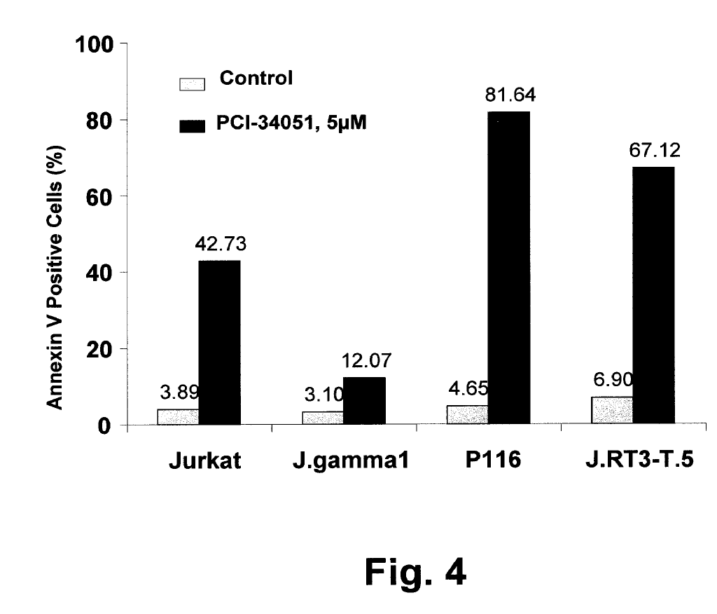

In order to further characterize the pro-apoptotic activity of PCI-34051, we tested its effect on Jurkat cells deficient in various steps of the T-cell receptor (TCR) signaling pathway. As shown in FIG. 4, and Table 1, PCI-34051 (5 μM), as well as a pan-HDAC inhibitor compound induced much less apoptosis in PLC-γ1-deficient Jurkat cells (J.γ1) than in wild type, TCR-deficient (J.RT3-T.5), or ZAP-70-deficient (P116) Jurkat cells.

TABLE 1
Apoptosis in WT and signaling mutant Jurkat cells

| | | | Pan-HDAC Inhibitor | | |
|---|---|---|---|---|---|
| | PCI-34051 | | Compound | | |
| 3 Day dose | GI50 | Apoptosis at | GI50 | Apoptosis at | |
| T-Cell line | (μM) | 5 μM (%) | (mM) | 0.125 mM (%) | Phenotype |
| Jurkat WT | 2.4 | 43 | 0.13 | 48 | Parent T-lymphocyte |
| J.g1 | 4.0 | 12 | 0.14 | 18 | Phospholipase C-γ1 |
| | | | | | deficient |
| P116 | 10.2 | 82 | 0.19 | 76 | ZAP-70 deficient |
| J.RT3-T.5 | 5.1 | 67 | 0.14 | 32 | TCR-b chain |
| | | | | | deficient |

This result suggested that PLC-γ1 signaling was necessary for the induction of apoptosis in T-cell lines by PCI-34051. Indeed, as shown in FIGS. 5-6, a PLC inhibitor (U-73122) inhibited PCI-34051-induced apoptosis in a dose-dependent manner. In contrast, an inactive analog of the PLC inhibitor (U-73343) failed to block PCI-34051-induced apoptosis.

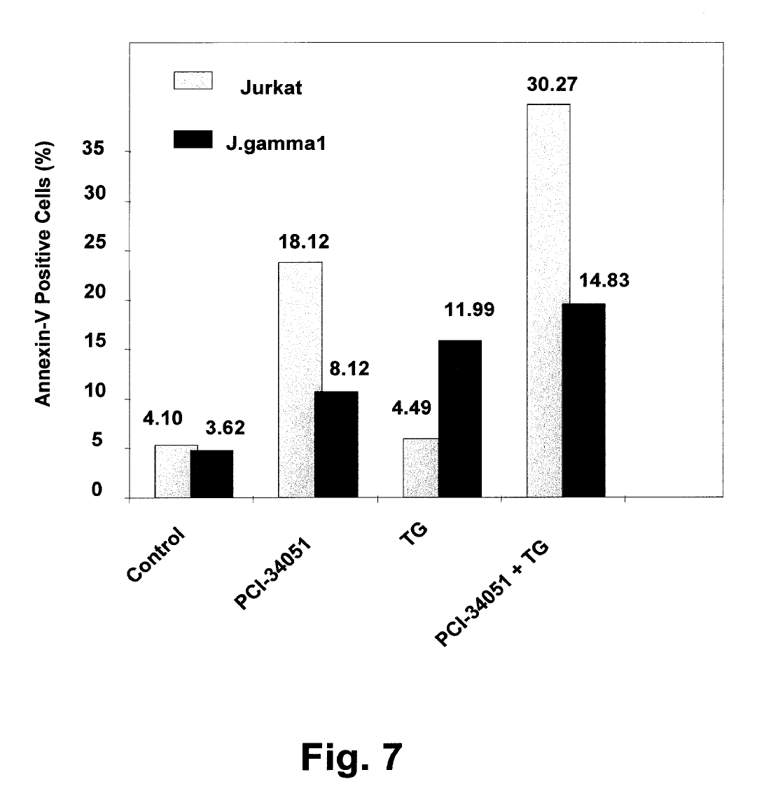

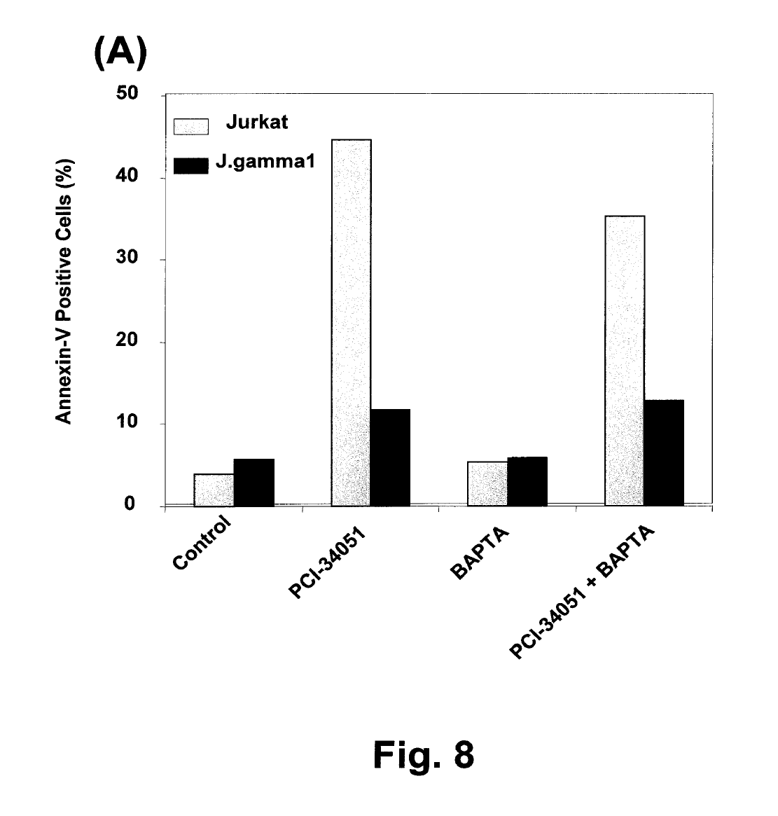

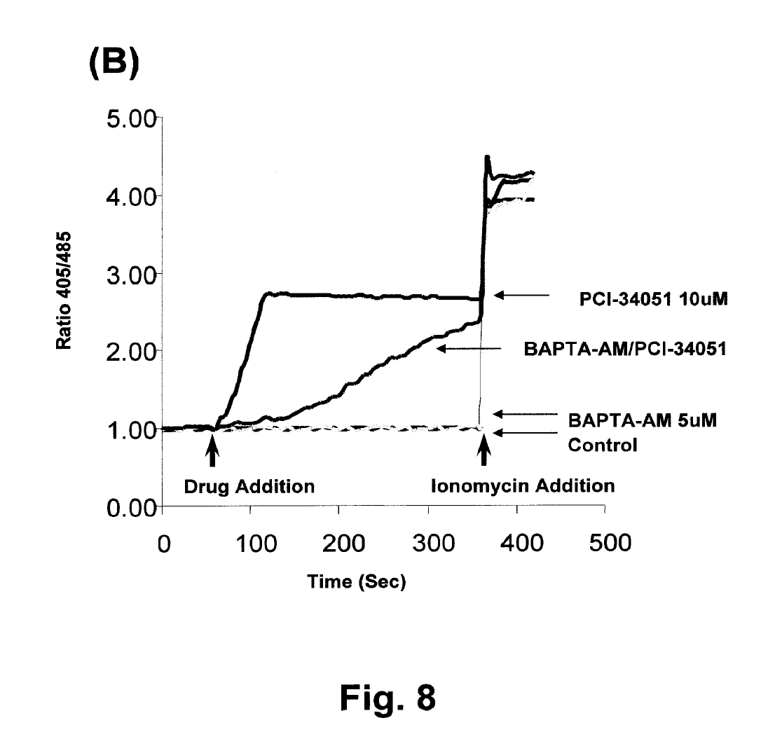

Consistent with the role of PLC in PCI-34051-induced apoptosis, we found that the Ca^{2α}-effector thapsigargin (0.2 μM) enhanced apoptosis, as shown in FIG. 7. In contrast, the Ca²⁺-chelator, BAPTA-AM (0.5 μM) diminished apoptosis induced by PCI-34051 as shown in FIG. 8. PCI-34051-induced calium flux in Jurkat cells was blocked by the calcium chelator BAPTA.

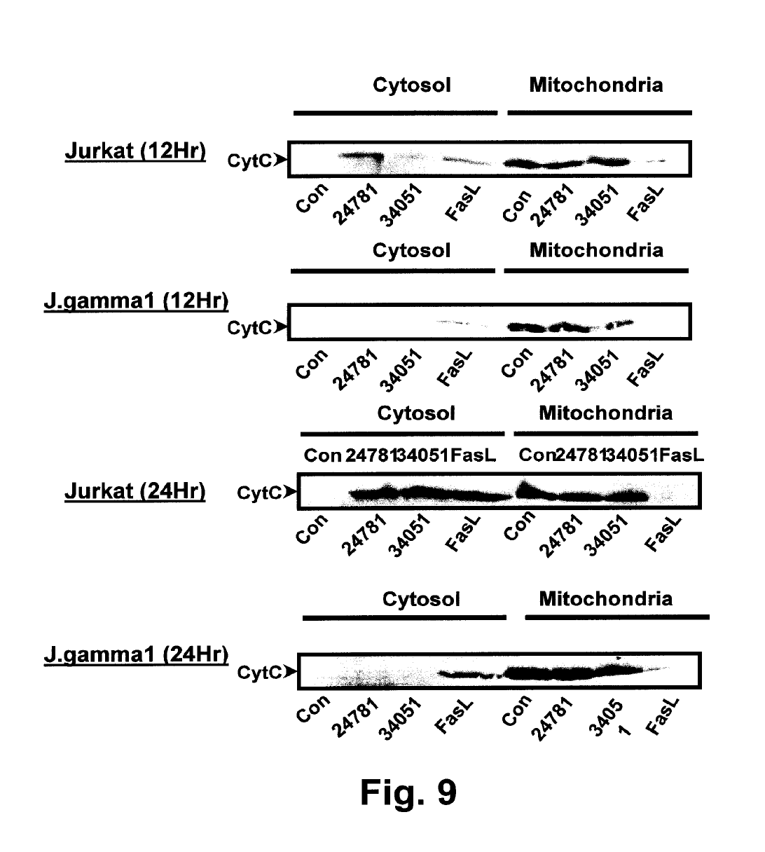

Finally, we examined cytochrome C translocation from mitochondria to cytosol, a hallmark of apoptosis, in response to PCI-34051 or a pan-HDAC inhibitor compound. As shown in FIG. 9 treatment with PCI-34051 or the pan-HDAC compound for 12 or 24 hours induced translocation of cytochrome oxidase from mitochondria to cytosol in wild type Jurkat cells. In contrast, the same treatments in the PLC-deficient J. cells, failed to alter the localization of cytochrome C. FasL, a pro-apoptotic protein, effectively induced translocation of cytochrome C in both WT and J.γ1 Jurkat cells.

Based on these results it was concluded that PCI-34051, an HDAC8-selective inhibitor compound induces apoptosis in T-cell derived lymphoma cells through a pathway that is dependent on the PLC signaling pathway. This suggests that activating the PLC signaling pathway is a useful therapeutic approach for treatment of T-cell proliferative disorders. Thus, HDAC inhibitor compounds (e.g., HDAC8-selective inhibitor compounds) alone or in combination with agents that activate PLC-dependent signaling (e.g., receptor agonists, receptor-activating, antibodies, thapsigargin, etc.) can be used to treat T-cell proliferative disorders. Conversely, profiling the PLC-signaling characteristics (e.g., PLC mRNA or protein levels, PLC enzymatic activity, or PLC-dependent changes in intracellular calcium levels) may be useful for determining cells likely to be responsive to an HDAC8-selective inhibitor.

### Example 5

### HDAC Inhibitors Induce PLC-γ Dependent Intracellular Calcium Mobilization

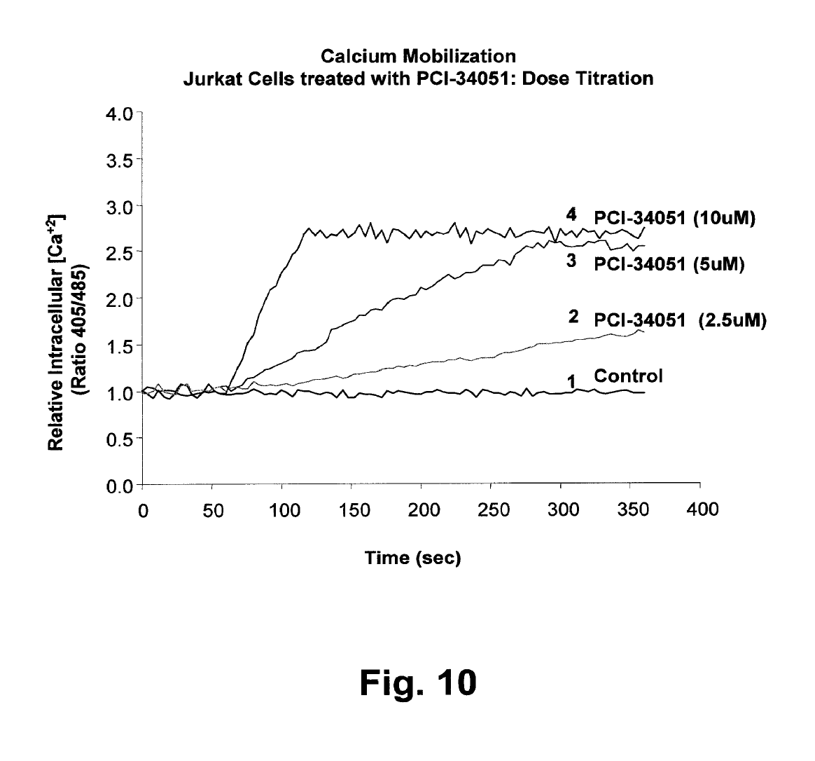

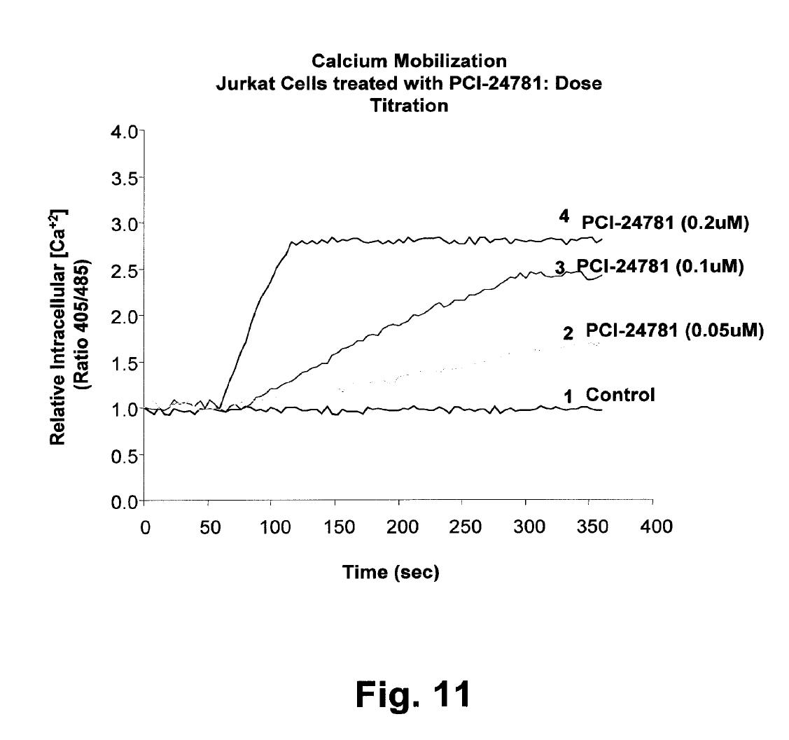

Ratiometric fluorescence calcium imaging was used to evaluate the effect of the HDAC-8 selective inhibitor compound, PCI-34051, and PCI-24781 (a pan-HDAC inhibitor compound) on intracellular calcium mobilization in T-cell and B-cell-derived cell lines. As shown in FIG. 2, the addition of 10 μM PCI-34051 or 0.2 μM PCI-24781 to cultured Jurkat cells, a T-cell derived cell line, resulted in a rapid (approximately 1 minute) and sustained rise in intracellular calcium very similar to that observed for the Ca²⁺-effector, thapsigargin (0.2 μM). As shown in FIGS. 5-6, the PCI-34051-stimulated increase in intracellular Ca²⁺ levels was strongly inhibited by the PLC inhibitor (U-73122), but not its inactive analog (U-73343), consistent with the effect of these compounds on apoptosis (Example 5). Further, the effect of either compound on intracellular Ca²⁺ was completely abolished in PLC-γ1-deficient HH cells (FIG. 3). Calcium mobilization by PCI-34051 and PCI-24781 in Jurkat cells was dose dependent, as shown in FIGS. 10 and 11, respectively. Interestingly, the HDAC8-selective compound PCI-34051 did not alter resting calcium levels in Ramos cells (FIG. 12(A)), which are B-cell derived. This result was consistent this compound's failure to induce apoptosis in this cell line. In contrast, the pan-HDAC inhibitor compound PCI-24781 induced a robust increase in intracellular calcium levels in this cell line (FIG. 12(A)). Importantly, Ramos cells do not express PLC-yl. Similarly, solid tumor cell lines such as the colon tumor line HCT-116 (which like B-cells contain active PLC-γ2) which are sensitive to PCI-24781 as measured by induction of apoptosis, also show calcium flux (FIG. 12 (C)), but PCI-34051 cannot induce either apoptosis or calcium flux. Finally, other solid tumor lines such as PC3 which do not show induction of apoptosis by either compound also do not show calcium flux (FIG. 12 (D)).

Two tumor cell lines (A549 lung tumor, THP-1 monocytic leukemia) were tested to correlate sensitivity of the pan-HDAC inhibitor, PCI-24781, and the HDAC8 inhibitor, PCI-34051, with the ability to undergo calcium flux. (FIGS. 13 and 14). The lung tumor line A549 is representative of an epithelial solid tumor line that responds to the pan-HDAC inhibitor by induction of apoptosis which is predicted by calcium flux, while the T-cell specific HDAC8 selective inhibitor PCI-34051 neither induces apoptosis nor calcium flux in these cells. Similarly THP-1 is a monocytic leukemia line in which the pan-HDAC inhibitor PCI-24781 can induce apoptosis and calcium flux, whereas the HDAC8 selective inhibitor PCI-34051 does not.

Based on these data we concluded that PCI-34051 likely exerts its effects (calcium mobilization and apoptosis) on T-cell derived cells selectively by acting through a PLC-γ1-dependent pathway, while PCI-24781 can induce apoptosis in tumor cells that contain either PLC-γ1 or PLC-γ2. In the case of either of these HDAC inhibitor (or any other HDAC inhibitor), the early calcium flux is a reliable predictor of apoptosis induced by that compound.

### Example 6

### Metabolites of HDAC Inhibitors Do Not Induce PLC-γ Dependent Intracellular Calcium Mobilization

Jurkat cells were tested to correlate sensitivity of the pan-HDAC inhibitor, PCI-24781, and two major metabolites of PCI-24781 (PCI-27789: carboxylic acid metabolite and PCI-27787: amide metabolite) with the ability to undergo calcium flux. (FIG. 15) Metabolites PCI-27789 and PCI-27787 have no histone deacetylase activity. PCI-27789 and PCI-27787 did not cause induction of calcium flux in Jurkat cells. Calcium flux was measured as described above in Example 1.

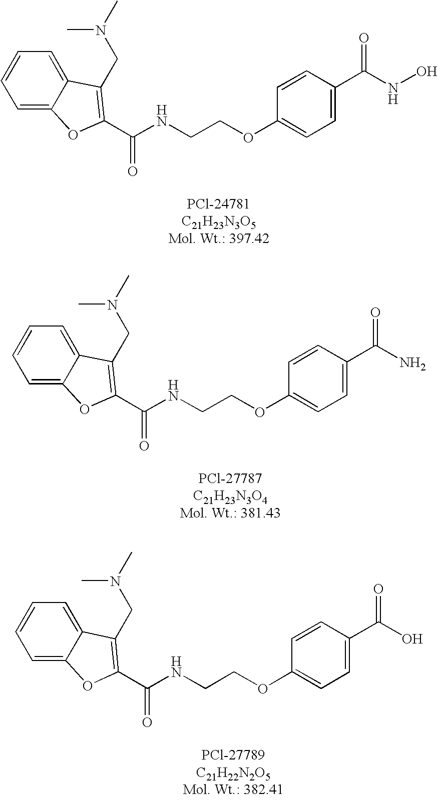

The two metabolites have almost exactly the same chemical structure as PCI-24781 and only differ from PCI-24781 in the “active” portion of the molecule: PCI-27789 has a carboxylic acid group instead of the hydroxamic acid group of PCI-24781; PCI-27787 has an amide group instead of the hydroxamic acid group of PCI-24781. The cell distinguishes these differences in structure in a matter of seconds (in terms of calcium flux), and signals whether or not to undergo apoptosis.

FIG. 16 shows induction of apoptosis by PCI-24781, while the inactive metabolites of PCI-24781, PCI-27789 and PCI-27787, have no apoptosis-inducing effect in Jurkat cells. Jurkat cells were treated with PCI-24781, PCI-27789 or PCI-27787 (0.2 uM) and apoptosis was measured (as described above in Example 2) by Annexin-V staining after 2 days. Thus, these figures are important in that they further establish a link between calcium flux and apoptosis.

### Example 7

### Positive and Negative Calcium Flux in a CTCL Patient Biopsy

FIG. 17 shows a positive calcium flux measured in a primary tumor biopsy from a patient with Cutaneous T-cell lymphoma (CTCL) (patient SF-03). The 5 mm punch biopsy was treated with collagenase, and incubated in growth media to which was added the pan-HDAC inhibitor, PCI-24781, or the HDAC8 inhibitor, PCI-34051. (Biopsies, i.e., cell extraction from primary tumors, were performed according to Example 3) Both compounds led to a rapid and significant calcium flux within 5 seconds of drug addition. Both compounds also led to an increase in tumor cell apoptosis following 40 hours of treatment.

Conversely, FIG. 18 shows a negative calcium flux measured in a primary tumor biopsy from another CTCL patient (patient SF-06). In this case, neither HDAC inhibitor stimulated calcium flux, nor did either HDAC inhibitor lead to apoptosis of the tumor cells. Thus, proving calcium flux is a rapid biomarker method to select and stratify patients in clinical trials who would respond to therapy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. (canceled)

3. (canceled)

4. (canceled)

5. (canceled)

6. (canceled)

7. (canceled)

8. (canceled)

9. (canceled)

10. A method of selecting a patient with a condition which responds to treatment with an apoptotic agent comprising the steps of:
(a) contacting a biological sample from a subject with a pan-HDAC inhibitor;
(b) measuring the level of calcium flux in the biological sample; and
(c) indicating that the patient should be selected for treatment with the apoptotic agent if the level of calcium flux observed in the biological sample following contact with the apoptotic agent exceeds the level of calcium flux observed in a control, or the biological sample before contact with the apoptotic agent; or
selecting an alternative treatment if the level of calcium flux observed in the biological sample following contact with the apoptotic agent does not exceed the level of calcium flux observed in a control or the biological sample before contact with the apoptotic agent.

11. The method of claim 10, wherein the level of calcium flux is measured with a calcium detection reagent.

12. The method of claim 11, wherein the calcium detection reagent is a fluorophore.

13. The method of claim 12, wherein the fluorophore is selected from the group consisting of: Fura-2, Indo-1, Fluo-3, calcein, Rhod-2, Rhod-4, and derivatives thereof.

14. The method of claim 10, wherein the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas.

15. The method of claim 10, wherein the biological sample comprises tumor cells.

16. The method of claim 15, wherein the biological sample comprises circulating tumor cells obtained from a blood sample.

17. The method of claim 15, wherein the biological sample comprises at least about 100 tumor cells.

18. A method of selecting a patient with a condition which responds to treatment with an apoptotic agent comprising the steps of:
(a) contacting a biological sample from a subject with an HDAC8 inhibitor;
(b) measuring the level of calcium flux in the biological sample; and
(c) indicating that the patient should be selected for treatment with the apoptotic agent if the level of calcium flux observed in the biological sample following contact with the apoptotic agent exceeds the level of calcium flux observed in a control, or the biological sample before contact with the apoptotic agent; or
selecting an alternative treatment if the level of calcium flux observed in the biological sample following contact with the apoptotic agent does not exceed the level of calcium flux observed in a control or the biological sample before contact with the apoptotic agent.

19. The method of claim 18, wherein the level of calcium flux is measured with a calcium detection reagent.

20. The method of claim 19, wherein the calcium detection reagent is a fluorophore.

21. The method of claim 20, wherein the fluorophore is selected from the group consisting ofL Fura-2, Indo-1, Fluo-3, calcein, Rhod-2, Rhod-4, and derivatives thereof.

22. The method of claim 18, wherein the condition is selected from the group consisting of: breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreas cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, chronic myelogenous leukemia, Burkitt's lymphoma, chronic myelogenous leukemia, H&N, Hodgkin's, CLL, B-cell lymphoma, and mantle and follicular cell lymphomas.

23. The method of claim 18, wherein the biological sample comprises tumor cells.

24. The method of claim 23, wherein the biological sample comprises circulating tumor cells obtained from a blood sample.

25. The method of claim 23, wherein the biological sample comprises at least about 100 tumor cells.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)
